(19) United States
(12) Patent Application Publication
(10) Pub. No.: US 2017/0269103 A1
(43) Pub. Date: Sep. 21, 2017

(54) Assessment of Heart Failure and Arrhythmic Risk Stratification by Measuring Circulating Hu Proteins

(71) Applicant: Rhode Island Hospital, Providence, RI (US)

(72) Inventor: Samuel C. Dudley, Providence, RI (US)

(21) Appl. No.: 15/505,020

(22) PCT Filed: Aug. 20, 2015
(86) PCT No.: PCT/US15/46162

§ 371 Date: Feb. 17, 2017

Related U.S. Application Data
(60) Provisional application No. 62/039,669, filed on Aug. 20, 2014.

(51) Int. Cl. G01N 33/68; C12Q 1/68

(52) U.S. Cl. CPC G01N 33/6893; C12Q 1/6883; G01N 2800/52; G01N 2800/326; G01N 2800/50; C12Q 2600/158; C12Q 2600/106

(57) ABSTRACT

This invention relates to a diagnostic test measuring circulating HU proteins or gene transcripts in a blood sample to assess arrhythmic risk in heart failure patients. The invention addresses this need and features a diagnostic/prognostic test for measuring circulating HU levels in a sample to assess arrhythmic risk in heart failure patients, alternatively, to determine the risk of SCD. The methods described herein represent a non-invasive (or minimally invasive) test assay. The methods described herein may also include computing a level of an SCN5A variant, or cardiac transcription factors MESP1 (mesoderm posterior posterior protein 1), or MEF2C (myocyte enhancer factor-2), or any combination thereof.

### CROSS-REFERENCE TO RELATED APPLICATIONS

This application which claims priority to U.S. Provisional Application No. 62/039,669, filed on Aug. 20, 2014, the contents of all of which are incorporated herein by reference in its entirety.

### STATEMENT AS TO FEDERALLY SPONSORED RESEARCH

This invention was made with government support under R01 HL1024025, P01 HL058000, R01 HL106592, Veteran Administration Merit Award, R41 HL112355, and P20 GM103652 awarded by National Institute of Health (NIH). The government has certain rights in the invention.

### SEQUENCE LISTING

Incorporated by reference in its entirety is a computer-readable nucleotide/amino acid sequence listing submitted concurrently herewith and identified as follows: 64 kilobyte Text file named “21486-620001WO_ST25_Sequence_Listing.txt,” created on Jul. 15, 2015.

### FIELD OF THE INVENTION

This invention relates to heart disease.

### BACKGROUND OF THE INVENTION

Heart failure (HF) represents a major health care concern in the United States. It has been estimated that approximately five million patients in the United States suffer from HF and an additional 500,000 patients are diagnosed with this disease every year. HF is therefore one of the most common diagnosis upon hospital admissions. HF patients have about a 6 to 9 fold greater risk of developing sudden cardiac death (SCD) compared to non-HF patients, thereby making SCD the leading cause of death for HF patients. Implanted cardiac defibrillators (ICDs) are used for the primary prevention of SCD in order to reduce total mortality in high risk HF patients. Unfortunately, results from clinical trials have not shown a statistically significant benefit for HF patients, in particular, in the highest 10-20% of predicted risk after ICD implantation. This is due in great part that the current SCD risk stratification techniques are not able to distinguish well amongst low and high arrhythmic risk patients, because in part, these tests do not reflect directly an arrhythmogenic pathophysiological process. Therefore, there is an unmet need for SCD risk assessment of HF patients.

### SUMMARY OF THE INVENTION

The invention addresses this need and features a diagnostic/prognostic test for measuring circulating HU levels in a sample to assess arrhythmic risk in heart failure patients, alternatively, to determine the risk of SCD. The protein is named HU, where “H” stands for histone and “U” stands for the 93U strain used initially to isolate the E. coli (Escherichia coli) nucleoid. HU is a small, basic, and thermostable dimeric DNA-binding protein, and is a major structural component of the nucleoid. Accordingly, a method for evaluating arrhythmic risk or assessing the severity of heart failure is carried out by using an HU binding agent to detect the level of an HU protein or an HU nucleic acid in a specimen from a subject. The method further comprises performing a reaction in vitro to yield a complex comprising the HU protein or nucleic acid and said binding agent, and detecting the level of the complex. A decrease in the level of the complex compared to a normal control indicates an increased, e.g., high risk, of arrhythmia or increased severity of heart failure. A decrease in the level of an HU protein or an HU nucleic acid compared to a normal control of at least 10%, 20%, 30%, 40%, 50%, or a 2-fold, 10-fold or greater decrease indicates a high risk of arrhythmia or an increased severity of heart failure.

The methods described herein represent a non-invasive (or minimally invasive) test assay. For example, the test sample such as blood is obtained by venipuncture, and the sample comprises circulating cells such as white cells, monocytes, T-cells or a bodily fluid such as blood, serum, or plasma. In another example, the test sample comprises saliva. In another example, the test sample comprises a buffy coat fraction. The buffy coat is a standard fraction of an anti-coagulated blood sample that contains most of the white blood cells and platelets following density gradient centrifugation of the blood.

Typically the subject is a human being characterized as comprising a risk of heart disease, e.g., a previous cardiac event, a family history of heart disease, or other risk factor such as obesity or diabetes. A diagnostic or prognostic level of HU protein or HU nucleic acid, e.g., cDNA an indicator of patient mRNA level, is a level that is decreased by at least 10% (at least 20%, 30%, 40%, 50% or more) compared to a normal control.

Human heart failure has been associated with reduced cardiac sodium channel current, in part because of a reduction in SCN5A mRNA abundance. Reduced SCN5A contributes to the arrhythmic risk in heart failure. The reduction in cardiac SCN5A mRNA abundance is reflected in circulating white cells that also express SCN5A. A diagnostic or prognostic level of a short variant form of SCN5A (e.g., a splice variant) is a level that is decreased by at least 10% (at least 20%, 30%, 40%, 50% or more) compared to a normal control as well. The presence of the variants caused the reduced abundance of the full-length SCN5A mRNA. The splice variant of the SCN5A gene is a splice variant produced from alternative splicing within Exon 28 of the SCN5A gene. In some aspects, the splice variant is a SCN5A Exon 28 B splice variant (a.k.a., E28B), a SCN5A Exon 28 C splice variant (a.k.a., E28C), or a SCN5A Exon 28 D splice variant (a.k.a., E28D). For example, the presence of one or more SCNA splice variants E28B, E28C and/or E28D in the biological sample identifies the subject as being at risk for developing arrhythmia (methods and compositions of SCN5A splice variants, e.g., SEQ ID NO. 7, 8, and 9 of U.S. application Ser. No. 13/291,826, and PCT application PCT/US2012/20564 and are incorporated therein in their entireties).

The methods described herein may also include computing a level of an SCN5A variant, or cardiac transcription factors MESP1 (mesoderm posterior posterior protein 1), or MEF2C (myocyte enhancer factor-2), or any combination thereof with a binding agent. Exemplary examples of a binding agent comprise an antibody (or fragment thereof), a detectable protein (or fragment thereof), a nucleic acid molecule such as one with a sequence that is complementary, such as an HU ligand, to patient mRNA or a cDNA produced from patient mRNA, or any combination thereof. The antibody may be labeled with a detectable moiety, e.g., a fluorescent compound or a radioactive agent (e.g., ¹²⁵I). When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The antibody can also be detectably labeled using fluorescence emitting metals such as 152 Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

According to the invention, a specific binding agent describes agents having greater than 10-fold, preferably greater than 100-fold, and most preferrably, greater than 1000-fold affinity for the target molecule as compared to another molecule. As the skilled artisan will appreciate the term specific is used to indicate that other biomolecules present in the sample do not significantly bind to the binding agent specific for the target molecule. Preferably, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, an antibody has a binding affinity in the low micromolar (10⁻⁶), nanomolar (10⁻⁷-10⁻⁹), with high affinity antibodies in the low nanomolar (10⁻⁹) or pico molar (10⁻¹²) range for its specific target ligand.

In one aspect, the invention describes a composition utilizing a binding agent, wherein the binding agent is attached to a solid support, (e.g., a strip, a polymer, a bead, or a nanoparticle). The strip may be a nucleic acid-probe coated porous or non-porous solid support strip comprising linking a nucleic acid probe to a carrier to prepare a conjugate and immobilizing the conjugate on a porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a binding agent (e.g., an antibody). Thus, the support configuration may be spherical, as in a bead, or cylindrical, as in the inside surface of a test tube, or the external surface of a rod. Alternatively, the surface may be flat such as a sheet, or test strip, etc. Preferred supports include polystyrene beads. Those skilled in the art will know many other suitable carriers for binding antibody or antigen, or will be able to ascertain the same by use of routine experimentation.

In other aspects, the solid support comprises a polymer, to which a agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device contains an HU binding agent alone or together with a binding agent for at least one, two, three or more other molecules, e.g., SCN5A.

The test is carried out on a bodily fluid or circulating cells such nucleated blood cells. In some cases, the cells, e.g., white blood cells, are lysed to yield a cell lysate prior to contacting the test sample (cell or cell lysate) with an HU binding agent. Detection is accomplished using an enzyme-linked immunosorbent assay (ELISA) or Western blot format. In other examples, the binding agent comprises a HU nucleic acid (e.g., primers or probe that are complementary for HU RNA or cDNA), and the detecting step is accomplished using a polymerase chain reaction (PCR) or Northern blot format, or other means of detection.

The term “sample” as used herein refers to a biological sample obtained for the purpose of evaluation in vitro. With regard to the methods disclosed herein, the sample or patient sample preferably may comprise any body fluid. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). In exemplary aspects, the sample comprises white blood cells obtained from the subject. In exemplary aspects, the sample comprises only white blood cells. In exemplary aspects, the sample is cardiac tissue (e.g., cardiac muscle tissue). Preferred samples are whole blood, serum, plasma, or urine.

Optionally, the method further comprises repeating the providing, contacting, detecting, and computing steps over time. A progressive decrease over time in the level of HU protein or HU nucleic acid indicates a progressive worsening of the severity of cardiac impairment, e.g., increased risk of arrhythmia, increased severity of heart failure, or increased risk of SCD. Optionally, the method may also include the step of treatment following risk stratification as described above. For example, the method further comprises identifying a subject with a high risk of arrhythmia or an increased severity of heart failure and administering to that subject a compound that increases HU protein or HU nucleic acid.

Thus, a method of treatment is also within the invention. A method of reducing risk of arrhythmia or severity of heart failure is carried out by identifying a patient characterized by a high risk base on a decreased level of HU protein or HU nucleic acid as described above and administering to the patient an HU elevating medication. For example, the HU elevating medication comprises an HU protein or fragment thereof, a nucleic acid encoding an HU protein or fragment thereof, e.g., using gene therapy. Other medications that improve the condition of the patient identified using the risk stratification methods described herein include administration of an antiarrhythmic drug, implanted cardioverter-defibrillator (ICD), angiotensin converting enzyme inhibitor (ACE), angiotensin II receptor blocker, beta-blocker, digoxin, diuretic, blood vessel dilator, aldactone inhibitor, or calcium channel blocker.

For purposes herein, the antiarrhythmic agent comprises any one of a group of pharmaceuticals that are used to suppress abnormal rhythms of the heart (cardiac arrhythmias), such as atrial fibrillation, atrial flutter, ventricular tachycardia, and ventricular fibrillation. In exemplary aspects, the anti-arrhythmic agent is a Singh Vaughan Williams (SVW) Class I, II, III, IV, or V anti-arrhythmic agent. In exemplary aspects, the antiarrhythmic agent is a SVW Class IA, IB, IC, or III anti-arrhythmic agent. The antiarrhythmic agent may be a fast-channel blocker, a beta blocker, a slow channel blocker, a sodium channel blocking agent, a potassium channel blocking agent, or a calcium channel blocking agent. The anti-arrhythmic agent in some aspects is one of Quinidine, Procainamide, Disopyramide, Lidocaine, Phenytoin, Mexiletine, Tocainide, Flecainide, Propafenone, Moricizine, Propranolol, Esmolol, Timolol, Metoprolol, Atenolol, Bisoprolol, Amiodarone, Sotalol, Ibutilide, Dofetilide, Dronedarone, E-4031, Verapamil, Diltiazem, Adenosine, Digoxin, or Magnesium Sulfate. In some aspects, the SVW Class IA is Quinidine, Procainamide, or Disopyramide. In some aspects, the SVW Class IB antiarrhythmic agent is Lidocaine, Phenytoin, Mexiletine, or Tocainide. In some aspects, the SVW Class IC anti-arrhythmic agent is Flecainide, Propafenone, Moricizine, or Encainide. In some aspects, the SVW Class III anti-arrhythmic agent is Dronedarone, Amiodarone, or Ibutilide. In some aspects, the anti-arrhythmic agent is NAD or mitoTEMPO.

Also within the invention is a kit comprising an HU binding agent and instructions for using the agent for evaluating arrhythmic risk or assessing the severity of heart failure. In some embodiments, the agent is attached to a solid support such a test strip. The kit optionally contains buffers, enzymes, salts, stabilizing agents, preservatives, and a container for receiving a patient test sample of bodily fluid or cell. In some cases such a container contains an anti-coagulant, cell separation agent (e.g., to separate white cells from red blood cells), or a cell lysis reagent, e.g., to liberate an HU protein or an HU nucleic acid such as mRNA from the cell to permit measurement of the protein or gene transcript). The agent may be attached to a solid support (e.g., a test strip). A complex intermediate of HU mRNA and test agent may be formed to detect such liberated HU nucleic acid. Still another embodiment of the invention is a kit comprising agents for measuring a group of markers, wherein the group of markers are defined as described in any of the preceding paragraphs, or panels containing figures, or other descriptions of preferred sets or panels of markers found herein. In some variations, such agents are packaged together. In some variations, the kit further includes an analysis tool for evaluating risk of an individual developing arrhythmia from measurements of the group of markers from at least one biological sample from the subject.

The diagnostic or prognostic assay is optionally formulated in a two-antibody binding format in which one HU protein-specific antibody captures an HU protein, e.g., HuR, in a patient sample and another HU-specific antibody is used to detect captured protein. For example, the capture antibody is immobilized on a solid phase, e.g., an assay plate, an assay well, a nitrocellulose membrane, a bead, a dipstick, or a component of an elution column. The second antibody, i.e., the detection antibody, is typically tagged with a detectable label such as a colorimetric agent or radioisotope.

The invention also describes diagnostic test system that obtains test results data representing levels of a marker in at least one biological sample. The results are collected and tracked and a means for computing an index value from said marker, wherein the index value comprises an arrhythmic risk score or a heart failure risk score, and a means of reporting the index value.

The invention can alternatively be defined as an improvement over existing methodologies, a method of evaluating the risk of developing an arrhythmia or heart failure in a subject. In an embodiment of the invention is an improvement comprising: obtaining marker measurement data that is representative of measurements of at least two markers in a sample from the subject, and evaluating the risk of developing arrhythmia or heart failure in the subject based on an output from a model, wherein the model is executed based on an input of the biomarker measurement data.

The following definitions are included for the purpose of understanding the present subject matter and for constructing the appended patent claims. Abbreviations used herein have their conventional meaning within the chemical and biological arts.

The term “about” refers to any minimal alteration in the concentration or amount of an agent that does not change the efficacy of the agent in preparation of a formulation and in treatment of a disease or disorder (e.g., arrhythmia). The term “about” with respect to concentration range of the agents (e.g., therapeutic/active agents) of the current disclosure also refers to any variation of a stated amount or range which would be an effective amount or range.

The term “activate” and its various noun and verbal permutations as used herein means increasing expansion, differentiation, or functional stimulation of cells. For example, helper T cells are activated to become effector cells. The helper T cells are activated on the surface of antigen-presenting cells, which mature during the innate immune responses triggered by an infection. The innate responses also dictate what kind of effector cell a helper T cell will develop into and thereby determine the nature of the adaptive immune response elicited.

The terms “administration” or “administering” refer to the act of providing an agent of the current embodiments or pharmaceutical composition including an agent of the current embodiments to the individual in need of treatment.

The singular forms “a,” “an,” and “the” include the plural reference unless the context clearly dictates otherwise.

The antibody is a polyclonal antisera or monoclonal antibody. The invention encompasses not only an intact monoclonal antibody, but also an immunologically-active antibody fragment, e. g., a Fab or (Fab)2 fragment; an engineered single chain FV molecule; or a chimeric molecule, e.g., an antibody which contains the binding specificity of one antibody, e.g., of murine origin, and the remaining portions of another antibody, e.g., of human origin.

The invention further comprises a humanized antibody, wherein the antibody is from a non-human species, whose protein sequence has been modified to increase their similarity to antibody variants produced naturally in humans. Generally, a humanized antibody has one or more amino acid residues introduced into it from a source which is non-human. These non-human amino acid residues are referred to herein as “import” residues, which are typically taken from an “import” antibody domain, particularly a variable domain.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

A “diagnostic test system,” means a system for obtaining test results data representing levels of multiple markers in at least one biological sample; means for collecting and tracking test results data for one or more individual biological samples; means for computing an index value from marker measurement data, wherein said biomarker measurement data is representative of measured levels of markers, wherein said measured levels of markers comprise the levels of a set or panel of markers; and means for reporting said index value. In some variations of the diagnostic test system, the index value is arrhythmia risk score. In some preferred variations, the arrhythmia risk score is computed according to the methods described herein for computing such scores. In some variations, the means for collecting and tracking test results data representing for one or more individuals comprises a data structure or database. In some variations, the means for computing arrhythmic risk score comprises a computer or microprocessor, comprising a visible display, an audio output, a link to a data structure or database, or a printer.

The term “evaluating arrhythmic risk” or “assessing the severity of heart failure” is used to indicate that the method according to the present invention will alone or together with other variables, (e.g., administration of an antiarrhythmic compound), establish or confirm the absence or presence of arrhythmia, or aid the physician in the prognosis, and or the monitoring of treatment. The skilled artisan will appreciate that any such evaluation or assessment is made in vitro. The patient sample is discarded afterwards. The patient sample is solely used for the in vitro diagnostic method of the invention and the material of the patient sample is not transferred back into the patient's body. Typically, the sample is a liquid sample, e.g., whole blood, serum or plasma.

By “fluoroimmunoassay” is meant an assay that has an agent labeled with a fluorophore.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules. cDNA is not naturally occurring.

By “mass spectrometry” is meant an analytical technique that helps identify the amount and type of components present in a sample by measuring the mass-to-charge ratio and the abundance of gas-phase ions. Exemplary techniques comprise electrospray ionization (ESI), matrix assisted laser desorption (MALDI), MALDI-TOF (Time of flight), Fourier transform ion cyclotron resonance (FTIC), and surface-enhanced laser desorption (SELDI).

The term “modulate”, “modulating” as used herein means regulating or adjusting to a certain degree.

The term, “normal amount” refers to a normal amount of a complex in an individual known not to be diagnosed with arrhythmia or heart failure. The amount of the protein can be measured in a test sample and compared to the “normal control level,” utilizing techniques such as reference limits, discrimination limits, or risk defining thresholds to define cutoff points and abnormal values (e.g., for arrhythmia). The normal control level means the level of one or more proteins or combined protein indices typically found in a subject known not suffering from arrhythmia. Such normal control levels and cutoff points may vary based on whether a protein is used alone or in a formula combining with other proteins into an index. Alternatively, the normal control level can be a database of protein patterns from previously tested subjects who did not convert to arrhythmia over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, BMI, current therapeutic regimen status, medical history, or a combination thereof, but differs from the subject being diagnosed in that the control does not suffer from the disease in question or is not at risk for the disease.

Relative to a control level, the level that is determined may an increased level. As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

As used herein, the term “nucleic acid” refers to polynucleotides such as deoxyribonucleic acid (DNA, e.g., cDNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

Polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

By “radioimmunoassay” (RIA) is meant as an in vitro assay used to measure concentrations of agents using an antibody, by use of labeled antigen (e.g., gamma-radioactive isotopes of iodine).

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Receiver-operating characteristics (ROC) describe the accuracy of a diagnostic method (See, Zweig, M. H., and Campbell, G., Clin. Chem. 39 (1993) 561-577). The ROC graph is a plot of all of the sensitivity/specificity pairs resulting from continuously varying the decision threshold over the entire range of data observed (also called a Youen's J Statistic or the Youden's Index). The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes (if the ROC plot falls completely below the 45° diagonal, this is easily remedied by reversing the criterion for “positivity” from “greater than” to “less than” or vice versa). Qualitatively, the closer the plot is to the upper left corner, the higher the overall accuracy of the test.

One preferred way to quantify the diagnostic accuracy of a laboratory test is to express its performance by a single number. Such an overall parameter, e.g., is the so-called “total error” or alternatively the “area under the curve=AUC”. The most common global measure is the area under the ROC plot. By convention, this area is always >0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

“Risk” in the context of the present invention, relates to the probability that an event will occur over a specific time period, as in the conversion to arrhythmia or heart failure, and can mean a subject's “absolute” risk or “relative” risk. A high risk subject may comprise an subject of developing arrhythmia or heart failure within 1 year. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

As used herein, the term “salt” refers to acid or base salts of the agents used herein. Illustrative but non-limiting examples of acceptable salts are mineral acid (hydrochloric acid, hydrobromic acid, phosphoric acid, sulfuric acid, and the like) salts, organic acid (acetic acid, propionic acid, glutamic acid, citric acid, and the like) salts, and quaternary ammonium (methyl iodide, ethyl iodide, and the like) salts.

The severity of a disease (e.g., arrhythmia or heart failure) may be expressed in terms of severity of symptoms that may be mild, severe or life-threatening. Common symptoms that are considered comprise chest pain, fainting, light-headedness, dizziness, paleness, shortness of breath, or sweating. Alternatively, arrhythmia may be quantified using a scoring method. For example, scores may correlate the incidences of ventricular fibrillation, ventricular tachycardia, and ventricular premature beats in early myocardial ischemia.

In certain embodiments, the invention provides methods for identifying a subject with a high risk of arrhythmia, or an increased severity of heart failure. Arrhythmias are more common in people who have diseases or conditions that weaken the heart (e.g., a heart attack, heart failure or cardiomyopathy) which weakens the heart and changes the way electrical signals move through the heart, heart tissue that is too thick or stiff or has not formed properly, leaking or narrowed heart valves, which make the heart work too hard and can lead to heart failure, congenital heart defects (defects present at birth) that affect the heart's structure or function, as well as conditions comprising high blood pressure, infections that damage the heart muscle or the sac around the heart, diabetes, which increases the risk of high blood pressure and coronary heart disease, sleep apnea, which can stress the heart because the heart doesn't get enough oxygen, an overactive or underactive thyroid gland (too much or too little thyroid hormone in the body). Several other examples of risk factors also can raise your risk for arrhythmias include heart surgery, certain drugs (such as cocaine or amphetamines), or an imbalance of chemicals or other substances (such as potassium) in the bloodstream.

The term “stabilizing agent” refers to a small molecule, an antibody (or fragment thereof), or a binding molecule that yields a complex that does not readily become inactive or denature, for example reversible (e.g., formaldehyde, SPDP (succinimidyl 3-(3-pyridyldithio)propionate)) and non-reversible cross-linkers.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated. With respect to a cell type, an isolated or purified cell is one that has been substantially separated or purified away from other biological components of the organism in which the cell naturally occurs, such as other cells of the organism. For example, an isolated lymphocyte cell population is a population of lymphocytes that is substantially separated or purified away from other blood cells, such as red blood cells. In a particular example, an isolated CD4 positive cell population is a population of CD4 positive cells that is substantially separated or purified away from other blood cells, such as CD8 positive cells. In one example, an isolated CD4 positive T-cell population is at least 95% pure, such as at least 99% pure. In another particular example, an isolated B-cell population is a population of B-cells that is substantially separated or purified away from other blood cells, such as T-cells. In one example, an isolated B-cell population is at least 95% pure, such as at least 99% pure. An enriched population of white blood cells (e.g., buffy coat fraction) is a population that have been separated from red blood cells, e.g., by density gradient.

As used herein, “treatment” is an approach for obtaining beneficial or desired results, including clinical results. For purposes of this disclosure, beneficial or desired clinical results include, but are not limited to, alleviation or amelioration of symptoms, diminishment of extent of disease, stabilized (i.e., not worsening) state of disease, preventing spread of disease, delay or slowing of disease progression, amelioration or palliation of the disease state, and recovery (whether partial or total), whether detectable or undetectable. “Treatment” can also mean prolonging survival as compared to expected survival if not receiving treatment.

Insofar as the methods of the present disclosure are directed to compositions and methods for treating a disease or disease state, it is understood that the term “prevent” does not require that the disease state (e.g., arrhythmia) be completely thwarted. The term “prevent” can encompass partial effects when the agents disclosed herein are administered as a prophylactic measure. The prophylactic measures include, without limitation, administration to one (or more) individual(s) who is suspected of being diagnosed with, e.g., arrhythmia.

Other features and advantages of the invention will be apparent from the following description of the preferred embodiments thereof, and from the claims. All references cited herein are hereby incorporated by reference.

### BRIEF DESCRIPTION OF THE DRAWINGS

FIG. 1A is a bar graph showing that levels of HuR mRNA decreased 38.9% in the ICD(+) event group compared with that in control (P=0.034). The expression of HuR and SCN5A mRNA in the white blood cells of normal control and heart failure patients with implanted cardioverter-defibrillators and evidence of appropriate event-driven therapy (ICD(+)) was determined.

FIG. 1B is a graph showing that SCN5A mRNA in the ICD(+) event group decreased 48.5% compared with that in control (P=0.001). The expression of HuR and SCN5A mRNA in the white blood cells of normal control and heart failure patients with implanted cardioverter-defibrillators and evidence of appropriate event-driven therapy (ICD(+)) was determined.

FIG. 2A is a schematic representation of the SCN5A mRNA ARE sites in its 3′-UTR. The length of the mRNA is indicated by a kilo base (kb)-scale.

FIG. 2B is a series of photographs of gel electrophoresis analysis and accompanying bar graph showing that HuR is the predominant member of Hu protein family expressed in human heart by both RT-PCR and RT-qPCR (percentages were normalized to the abundance of HuR mRNA, which was set up as 100%).

FIG. 2C is a photograph of a gel electrophoresis analysis showing SCN5A mRNA was associated with HuR in a RNA immunoprecipitation assay. RT indicates reverse transcription.

FIG. 3A is a bar graph illustrating the effect of ectopic overexpression of HuR on SCN5A mRNA expression. Human cardiomyocytes were transfected with a mammalian expression vector encoding HuR cDNA or control vector lacking HuR for 48 h. Values are the means±SEM from triplicate experiments. Overexpression of HuR increased SCN5A mRNA by 46.3% compared with control group (P=0.04).

FIG. 3B is a photograph of a gel electrophoresis analysis showing Western blots overexpressing HuR using an anti-HA antibody.

FIG. 3C is a graph showing that SCN5A mRNA was more stable in HuR transfected cells than that in control cells. Cells were transfected for 48 h with HuR or a control vector before transcription. Transcription was blocked by the addition of actinomycin D (5 μg/ml). After the indicated time periods, cells were harvested and extracted for total RNA. The graph shows the percentage of remaining SCN5A mRNA levels in relation to β-actin mRNA and compared with the levels of normalized mRNA species measured immediately before the addition of actinomycin D, which was set to 100%.

FIG. 4A is a bar graph showing that the silencing of HuR by siRNA decreases SCN5A mRNA levels. Cells were transfected with either siRNA duplexes of HuR (siHuR) or control-siRNA duplexes (siCtrl). Cells were harvested and extracted for total cellular RNA 48 h after transfection, and changes in SCN5A and HuR mRNA levels were determined by RT-qPCR, and using β-actin mRNA for normalization. SCN5A mRNA decreased 32.5% (P=0.001) in cells transfected with siRNA of HuR (siHuR) compared with the level of corresponding mRNA in those transfected with scramble siRNA (siCtrl).

FIG. 4B is a bar graph showing that the silencing of HuR by siRNA decreases SCN5A mRNA levels. Cells were transfected with either siRNA duplexes of HuR (siHuR) or control-siRNA duplexes (siCtrl). Cells were harvested and extracted for total cellular RNA 48 h after transfection, and changes in SCN5A and HuR mRNA levels were determined by RT-qPCR, and using β-actin mRNA for normalization. HuR mRNA decreased 77.3% in cells with siHuR (P=0.000).

FIG. 5 is a graph illustrating receiver operation characteristics (ROC) curves for WBC HuR discrimination of ICD events. The area under the ROC curve was 0.93 (with a 95% CI (confidence interval of 0.78-1.09) for HuR, P=0.018.

FIG. 6 is a table showing optimal discrimination values for HuR mRNA.

FIG. 7A is a graph showing the expression of SCN5A in WBCs of a normal control (N=5) and BrS Patients (N=25). Levels of SCN5A mRNA decreased 58.04% (P=0.01) in BrS patients (0.62±0.62) compared with that in the control subjects (1.43±0.59). Among the 25 BrS patients, 20 patients have known SCN5A mutations, and 5 patients lack identified SCN5A mutations.

FIG. 7B is a graph showing the expression of HuR in WBCs of a normal control (N=5) and BrS (Brugada Syndrome Patients (N=25). HuR mRNA of BrS patients (0.56±0.26) was 45.63% (P=0.001) lower than that of normal control patients (1.03±0.28).

FIG. 7C is a graph showing the expression of MEF2C in WBCs of a control (N=5) and BrS Patients (N=25). MEF2C mRNA of BrS group (0.54±0.30) decreased 41.3% (P=0.02) compared with that in normal control group (0.92±0.42).

FIG. 8A is a graph showing that the expression of SCN5A correlates with the expression of HuR in WBCs. (R²=0.402, P=0.000).

FIG. 8B is a graph showing that the expression of MEF2C mRNA correlates with the expression of SCN5A in WBCs (R²=0.268, P=0.003).

FIG. 9A is a bar graph showing that MEF2C regulates SCN5A expression in cardiomyocytes. MEF2C increased SCN5A-mRNA expression in doxycycline dose-dependent manner.

FIG. 9B is a bar graph showing SCN5A mRNA decreased 22.3% (P=0.001) in cells transfected with siRNA of MEF2C (siMEF2C) compared within those transfected with scramble siRNA (siCtrl).

### DETAILED DESCRIPTION OF THE INVENTION

### Heart Failure

Heart failure (HF) is defined as the ability of the heart to supply sufficient blood flow to meet the body's needs. The signs and symptoms of heart failure include dyspnea (e.g., orthopnea, paroxysmal nocturnal dyspnea), coughing, cardiac asthma, wheezing, dizziness, confusion, cool extremities at rest, chronic venous congestion, ankle swelling, peripheral edema or anasarca, nocturia, ascites, heptomegaly, jaundice, coagulopathy, fatigue, exercise intolerance, jugular venous distension, pulmonary rales, peripheral edema, pulmonary vascular redistribution, interstitial edema, pleural effusions, or a combination thereof. A symptom of heart failure may be one of the symptoms listed in the following table, which provides a basis for classification of heart failure according to the New York Heart Association (NYHA).

| NYHA | |
|---|---|
| Class | Symptoms |
| I | No symptoms and no limitation in ordinary physical activity, |
| | e.g., shortness of breath when walking, climbing stairs, etc. |
| II | Mild symptoms (mild shortness of breath and/or angina) and |
| | slight limitation during ordinary activity |
| III | Marked limitation in activity due to symptoms, even during |
| | less-than-ordinary activity, e.g., walking short distances |
| | (20-100 m). Comfortable only at rest |
| IV | Severe limitations. Experiences symptoms even while at rest. |
| | Mostly bedbound patients. |

The heart failure may be a systolic heart failure, which is heart failure caused or characterized by a systolic dysfunction. Systolic dysfunction is a condition in which the pump function or contraction of the heart (i.e., systole), fails. Systolic dysfunction is characterized by a decreased or reduced ejection fraction, e.g., an ejection fraction which is less than 45%, and an increased ventricular end-diastolic pressure and volume. In some aspects, the strength of ventricular contraction is weakened and insufficient for creating an appropriate stroke volume, resulting in less cardiac output. Systolic heart failure includes ischemic heart failure, or nonischemic heart failure.

Heart failure is associated with upregulation of two cardiac SCN5A mRNA splice variants that encode prematurely truncated, nonfunctional Na⁺ channels (U.S. Patent Publication No. 20120129179, hereby incorporated by reference). The methods described herein are based on the observation that a decrease of HuR mRNA correlated with a reduction of wild-type SCN5A mRNA expression in heart failure patients.

### Arrhythmias

The invention moreover provides a method of determining a subject's risk for arrhythmia. As used herein, the term “arrhythmia” is synonymous with “cardiac dysrhythmia” or “cardiac arrhythmia” and refers to any condition in which there is abnormal electrical activity in the heart. The cardiac arrhythmia may be a ventricular arrhythmia, such as ventricular fibrillation, ventricular tachycardia, or an arrhythmic condition in which both ventricular fibrillation and ventricular tachycardia are present. The cardiac arrhythmia may be an atrial arrhythmia, e.g., an atrial fibrillation, atrial tachycardia, or an arrhythmic condition in which both atrial fibrillation and atrial tachycardia are present. Other types of cardiac arrhythmias are described below. The cardiac arrhythmia may be characterized by an abnormal heart rate, such as a bradycardia or a tachycardia.

### Bradycardia

A bradycardia is a cardiac arrhythmia in which the resting heart rate is slower than normal, and is characterized by a resting heart rate in an adult human which is slower than 60 beats per minute. The bradycardia is a sinus bradycardia caused by sinus arrest or AV (atrioventricular) block or heart block. Alternatively, the bradycardia is caused by a slowed electrical conduction in the heart.

### Tachycardia

A tachycardia is a cardiac arrhythmia in which the resting heart rate is faster than normal, which is faster than 100 beats per minute. The tachycardia may be a sinus tachycardia, and is not caused by physical exercise, emotional stress, hyperthyroidism, ingestion or injection of substances, such as caffeine or amphetamines. Alternatively, the tachycardia may not be a sinus tachycardia, e.g., a tachycardia resulting from automaticity, reentry (e.g., fibrillation), or triggered activity. The tachycardia may be caused by a slowed electrical conduction in the heart, an ectopic focus, or is combined with abnormal rhythm.

### Brugada Syndrome BrS

Brugada Syndrome (BrS) is an inherited sudden death condition caused mostly by reductions in cardiac sodium current. The condition arises when sodium channels behave abnormally, wherein the movement of sodium ions into the cells is restricted which results in changes in the ECG (electrocardiogram), with no structural abnormalities. SCN5A was the first gene known to be associated with BrS. About 20% of BrS patients are thought to carry mutations in SCN5A gene and mutations in more than 10 genes have been reported to cause BrS. Nevertheless, approximately 80% of clinically diagnosed BrS patients do not carry any mutation known to cause BrS. Sodium channel transcript levels have been shown to correlate between heart and white cells, indicating that white cells are useful to assess cardiac SCN5A transcription. Therefore, measurement of white blood cell SCN5A expression levels may reveal cardiac sodium channel expression changes in BrS patients.

### RNA-Binding Proteins

RNA-binding proteins (RBPs) play an essential role in the maturation and function of mRNAs. RBPs have crucial roles in various cellular processes such as cellular function, transport and localization, as well as post-transcriptional control of RNAs, such as splicing, polyadenylation, mRNA stabilization, mRNA localization and translation. RBPs exhibit highly specific recognition of their RNA targets by recognizing their sequences and structures. Specific binding of the RBPs allows them to distinguish their targets and regulate a variety of cellular functions via control of the generation, maturation and lifespan of the RNA transcript. Exemplary RBPs include the HU family of proteins.

### HU Proteins

HU proteins are RNA-binding proteins involved in diverse biological processes, such as neuronal development and cellular stress response. HU proteins affect the expression of their regulons through diverse mechanisms, from splicing to translation, e.g., their ability to stabilize target mRNA by binding to AREs in their 3′ untranslated regions. HU proteins recognize and bind to AU-rich RNA elements (AREs) and also show an empirical preference for U-rich sequences as well as some other RNA sequences. Each HU protein has three RNA recognition motifs (RRMs 1-3), which share more than 90% amino acid sequence identity among family members. The SCN5A mRNA 3′ untranslated region (UTR) was shown to contain two sets of AU-rich elements (ARE), which may be able to bind RNA-binding proteins such as HU proteins.

Additionally, RBPs may regulate the expression of multiple mRNAs that encode functionally related proteins, termed RNA operons. Individual mRNAs can be members of multiple operons, forming higher-order “RNA regulons.” Thus, as RBPs, HU proteins may perform their overall biological functions by coordinately regulating functionally related mRNAs. All of the biological functions of HU proteins are believed to be a result of their ability to bind specific target mRNAs and affect their expression. In the cytoplasm, HU proteins are best known for stabilizing target mRNAs, (for example, GAP-43, VEGF, and GLUT1), by binding to AREs in their 3′ untranslated regions (UTRs) and prevent their degradation, and thus indirectly enhancing protein production.

Although HU proteins are best known for stabilizing mRNAs, they can also affect target protein expression at the level of translation. Unlike their role in mRNA stability in which target protein expression is enhanced, HU proteins may act as enhancers or repressors of translation. HU proteins upregulate the translation of many target mRNAs, which result in increased recruitment of target mRNAs to polysomes, indicating increased translation initiation. In the nucleus, HU proteins serve as regulators of polyadenylation and alternative splicing.

The HU proteins: HuA (HuR in Human), HuB (HelN1 in Human), HuC, and HuD are a family of mammalian RNA-binding proteins (Zhu et al., 2006, Mol. Biol. Cell. 17: 5105-5114). Of the four Hu family members, HuA/R is widely expressed in many cell types, whereas HuB, HuC, and HuD, are expressed specifically in neurons. In addition to HuR (ELAVL1, GenBank accession number: NM_001419.2), the ELAV/HU family members HuB (ELAVL2, GenBank accession number: NM_004432.3), HuC (ELAVL3, GenBank accession number: NM_001420.3) and HuD (ELAVL4, GenBank accession number: NM_001144774.1) are also useful diagnostic or prognostic markers.

### HuR and HuB Protein

The family HU family member, HuR, has a variety of biological functions. Through its post-transcriptional regulation of targets, such as several genes controlling cell growth and proliferation, HuR is believed to mediate cellular response to DNA damage and other types of stress.

The initiator methionine of HuR is removed in the mature processed form yielding a mature protein of 325 amino acids. HuR contains three RNA-recognition motif (RRM) domains. The RRM1 domain comprises residues 20-98 (79 amino acids in length); RRM2 domain comprises residues 106-186 (81 amino acids in length); and the RRM3 domain comprises residues 244-322 (79 amino acids in length). Fragments of HuR, such as fragments corresponding to the domains described above, are useful in the compositions and methods described herein. The protein is characterized by the following amino acid modifications: Modified residue 2-N-acetylserine; modified residue 2-Phosphoserine; modified residue 202-Phosphoserine, and/or modified residue 217-Omega-N-methylated arginine. This encoded protein contains 3 RNA-binding domains and binds cis-acting AU-rich elements. It stabilizes mRNAs and thereby regulates gene expression.

### Human HuR Amino Acid Sequences

| | |
|---|---|
| 1 | msngyedhma edcrgdigrt nlivnylpqn mtqdelrslf ssigevesak lirdkvaghs |
| 61 | lgygfvnyvt akdaeraint lnglrlqskt ikvsyarpss evikdanlyi sglprtmtqk |
| 121 | dvedmfsrfg riinsrvlvd qttglsrgva firfdkrsea eeaitsfngh kppgssepit |
| 181 | vkfaanpnqn knvallsqly hsparrfggp vhhqaqrfrf spmgvdhmsg lsgvnvpgna |
| 241 | ssgwcifiyn lgqdadegil wqmfgpfgav tnvkvirdfn tnkckgfgfv tmtnyeeaam |
| 301 | aiaslngyrl gdkilqvsfk tnkshk |

(SEQ ID NO: 1) GenBank Accession NP_001410.2 (GI: 38201714), incorporated herein by reference.

Exemplary landmark residues, domains, and fragments of HuR include residues 243-326 (the RNA recognition motif 3 in vertebrate Hu-antigen R), residues 180-183 (β-strand region), residues 157-167 (helical region), residues 146-156 (β-strand region), residues 141-143 (hydrogen bonded turn), residues 107-185 (RNA binding site), residues 106-189 (RNA recognition motif), residues 19-96 (RNA recognition motif 1), residues 19-326 (ELAV/HuD family splicing factor). A fragment of an HU protein is less than the length of the full length protein, e.g., a fragment is 10, 20, 30, 40, 50, 100, 200 or more residues in length, but less than e.g., 326 residues in the case of HuR above.

Human HuR nucleotide sequence. The start and stop codons of the coding sequence are bold and underlined.

| | |
|---|---|
| 1 | ggtcgtgcgc gctgaggagg agccgctgcc gccgtcgccg tcgccgccac cgccgccacc |
| 61 | gctaccgagg ccgagcggag ccgttagcgc cgcgccgccg ccgcctcccg cccgccccgg |
| 121 | agcagccccg ggcccgcccg cccgcatcca gatttttgaa aaatacaatg tctaatggtt |
| 181 | atgaagacca catggccgaa gactgcaggg gtgacatcgg gagaacgaat ttgatcgtca |
| 241 | actacctccc tcagaacatg acccaggatg agttacgaag cctgttcagc agcattggtg |
| 301 | aagttgaatc tgcaaaactt attcgggata aagtagcagg acacagcttg ggctatggct |
| 361 | ttgtgaacta cgtgaccgcg aaggatgcag agagagcgat caacacgctg aacggcttga |
| 421 | ggctccagtc aaaaaccatt aaggtgtcgt atgctcgccc gagctcagag gtgatcaaag |
| 481 | acgccaactt gtacatcagc gggctcccgc ggaccatgac ccagaaggac gtagaagaca |
| 541 | tgttctctcg gtttgggcgg atcatcaact cgcgggtcct cgtggatcag actacaggtt |
| 601 | tgtccagagg ggttgcgttt atccggtttg acaaacggtc ggaggcagaa gaggcaatta |
| 661 | ccagtttcaa tggtcataaa cccccaggtt cctctgagcc catcacagtg aagtttgcag |
| 721 | ccaaccccaa ccagaacaaa aacgtggcac tcctctcgca gctgtaccac tcgccagcgc |
| 781 | gacggttcgg aggccccgtt caccaccagg cgcagagatt caggttctcc cccatgggcg |
| 841 | tcgatcacat gagcgggctc tctggcgtca acgtgccagg aaacgcctcc tccggctggt |
| 901 | gcattttcat ctacaacctg gggcaggatg ccgacgaggg gatcctctgg cagatgtttg |
| 961 | ggccgtttgg tgccgtcacc aatgtgaaag tgatccgcga cttcaacacc aacaagtgca |
| 1021 | aagggtttgg ctttgtgacc atgacaaact atgaagaagc cgcgatggcc atagccagcc |
| 1081 | tgaacggcta ccgcctgggg gacaaaatct tacaggtttc cttcaaaacc aacaagtccc |
| 1141 | acaaataact cgctcatgct tttttttgta cggaatagat aattaagagt gaaggagttg |
| 1201 | aaacttttct tgttagtgta caactcattt tgcgccaatt ttcacaagtg tttgtctttg |
| 1261 | tctgaatgag aagtgagaag gtttttatac tctgggatgc aaccgacatg ttcaaatgtt |
| 1321 | tgaaatccca caatgttaga ccaatcttaa gtttcgtaag ttatttcctt taagatatat |
| 1381 | attaaacaga aatctaagta gaactgcatt gactaaccag tccctctgga tggtggtgaa |
| 1441 | cctgaagcat gctttaacct ctaagactgt ctaacacgcg tttcattcaa tgtctccaca |
| 1501 | gactgggtag caaaaaaatc accttttagt tttagttttt aatctaaaga tgttagacag |
| 1561 | atgctgagtg tgcgttttct caaccgcttc aacattgtaa gcgatgtatg ctttggttga |
| 1621 | caggaagttc cttttccagg caggtcccgt tgccacctcc tgctcactca gtcccgggct |
| 1681 | ctgccgagtg gtcctgggaa tggcggcggg cccgtccagc gtgggccacc actggggccg |
| 1741 | ggggccacgg gctgcatgct gggcgggccc tccagagaag gacacaaacg tgtttcgtaa |
| 1801 | gcccaggcac caatgggaat ggaccaaaga gtttcaggga aactccagta tattccagag |
| 1861 | tcagatctaa gctccaggca cgcctgaaga tgtgttgcta ctctgacatc ccgagtttct |
| 1921 | gtccacacat tgcatgcaca gcgccccaca cattggatac tgttgttcac gataatttct |
| 1981 | cccgttttcc agagcattta acatagcttg gaggcgtaaa atggctctgt attttaataa |
| 2041 | cacagaaaca tttgagcatt gtatttctcg catcccttct cgtgagcgct tagacctttt |
| 2101 | tctattttag tcggattttg ttttggaatt ttgcttttgt atgaacactc agcagaaaag |
| 2161 | tacttacttc ttgccagtta tctattaacc aaaacctttg atttgtagtt ttaaagatta |
| 2221 | accctcaaag ttctcttcat aactgccttg acattttggg ttgttctgtt ctgttaattt |
| 2281 | tcttttgctt ttttgtgttt tttgtttgtt tttacttttg catttaagac cattaaattt |
| 2341 | gattttgttt tgctcgaatt ttgttttgtt ttttatttta ccttttcttt ctttttggct |
| 2401 | agggaaggtg caggtggccc agcattcagg gaggagtcgt aagatcttaa gaaaccaatc |
| 2461 | cttgcctcaa gcaaaagcat ttctgaatct gtgacgcaag aatgtgcagt tacaggctgg |
| 2521 | tggcttttaa accaggagcc cggaggaagg gtgaaagaga aagcctgtga aataggcagg |
| 2581 | gccagatcac ccaaaactcc tcaggactgg gatctggcgt ttataaataa ctagtttaca |
| 2641 | gagagaatca caaacaggat aacttagtac cagcagtttt taaccttgac gtgagactaa |
| 2701 | aacgtgaccg taggctgttt tttagttatt gctctcatga gatgatggct gtatttatct |
| 2761 | gtttatttat acctatttat gtatttattt attgaagtgt gaaattgagc aataggcagg |
| 2821 | caccaccgtc cccagagcag gtcagcgtct cgagaggccc ctggacaatt gaggatgccc |
| 2881 | atcccctccc ctttccctga tcttttactg aggggctgtg tgcgcgatcc ttgcaaattg |
| 2941 | atgatgttgc catccgtacc caggctgtgt ctcataaaag tcggcctggt gccagagagg |
| 3001 | acctcctttc tcccacagaa tcccagatcc tcaggaaaag ccaaaaccga ggcccattgc |
| 3061 | ccggattcga cacaaaagag ggtccctgct ctgttgcccg agagcagtct gcatcctggg |
| 3121 | accagaatgc tttcctggaa aaagaagcct ttcaggtttc cctgggccag catcttctga |
| 3181 | tggaaggtgg gagccaacac ccttctgatg gaaggtggga gccaacaccc ttctgatgga |
| 3241 | aggtgggagc caacaccctt ctgatggaag gtgggagcca acacccttct gatggaaggt |
| 3301 | gggagccaac acccttctga tggaaggcgg gattcccgct tctgaaactc cccctggagt |
| 3361 | ctcactccca cacatgccca tagctagcat tcaacagaga actctgtctt aagcttcaac |
| 3421 | tgtgaaaatg atgacgggct tgtagcacct cagcttcttt cctcgccccc ttttatctga |
| 3481 | atcctatcaa ttattctgat gctgggacag gtgagaagaa actgtgaagt atatgagcct |
| 3541 | ttgaaagttc cctgaagttt ctcagttcag gaacattctc attgtatgtg gtctccgctg |
| 3601 | tttgaacagc cttctagcta aaaaattcca aagcctttat ttgggagtct tagcttgcaa |
| 3661 | gcttgtggaa ggatttagct taacaactgt cactcctgaa aagcaatctc tgttccatca |
| 3721 | aggttctagt tgctggccct gtgtcctcaa agttcattac atcttatcaa ggcctgtttg |
| 3781 | caaaggggag atcccttttc ttaaaaaagg ctcaacccaa aagaaaccat ttcttaaaaa |
| 3841 | attttacata gatcagttgt atttctattt agcaaaaatg agtgctctgc ttttatttgg |
| 3901 | gaatttcgat gaaaaagcgt tcagagtaga taatgttcat ttatcaaaaa tctggtttgg |
| 3961 | gaaataccaa agaggctttg attgaattcc cttttgaccc gtgtgtaact tcctctggta |
| 4021 | gttagacccc aggcagctcc gaatttgtga acctgcttcc tgatgaattc tcccttgttc |
| 4081 | ccccttggct ctgccattat ttcgttttca gtgtaatttg ccaagccgca gttttctgtg |
| 4141 | ctggctgtgc ctctagtcgc agctctgtga ctgattccct cccgggtgct gagtcccctc |
| 4201 | cccggccacc atcctgcgtg aatatcctga aattcatggg cttcctcggg ggcccgcccg |
| 4261 | caggtggtgc tgggtgggtt ccgccacctt ctcctggaag gtgagccttt tcctggccaa |
| 4321 | gggcagctgc cttaacctct gagagtctgc gcttggcctt agtcctggag acccagcctc |
| 4381 | cagggactga accgtgctgc tgttgggagc caagaccggc cctttggagc cggcagccca |
| 4441 | ggggtccctg ctggatcaga gaaatagaag cacccgaaga cggttagtgg caattccttg |
| 4501 | acccggtttg cttccaaatg aaggccattt gtccaccagg cattgaaaag acatgactta |
| 4561 | cccagtccgg catcggactt gaaaaatcga aattgacatc actcagctgt tacatttcac |
| 4621 | atccgattca gccccctttt atttccatgt gcttttcgca gccttcctgt gttggatgaa |
| 4681 | agagaataag aattcagctg acaggaggcc tctatcctgt ccctccaccc caccctccac |
| 4741 | ctcaatcccc tcccatcttc cccagaccta cctcacctac taggacctga ggcagctcct |
| 4801 | tagcagagac ccctggggtt tagctgactc tgggggtcag gggttcctgt ccccaaactt |
| 4861 | cgcaagacag ccctgaagtc acaagtgctt tcttttaagt ggcattggca attggcgtgt |
| 4921 | aatgatggca gtagaatctg aatctcggat cccaggcagg gttcacattt ccaaaccttt |
| 4981 | ttgatttccc ctgacctcta atggctggat cctatttttc tacaaccttt cagtgacatc |
| 5041 | gttcaggttt ctttcttggc catttaaaaa aacaaatttt ttttttctca cttgtaagtc |
| 5101 | accgccagta cctaagttag gctaacggag actttgacag gactggattt ttcttccacc |
| 5161 | agaagagaag ccttttccgt tggtttgggg ccacctcttt gaccatgacc atgtgatgtt |
| 5221 | ccgtttacag tgacttgctt tgggggaggg gaggctctct taaccgattc ccatgttgta |
| 5281 | cagtagatgg ttagaccttt tgtatattag tgtgttttaa gttattgatt tgttttatat |
| 5341 | aaaataattt atttttcagg tgccattttt cattttaact ttgtttttac atgggtttgt |
| 5401 | tttcaataaa gtctgacact ggtgtccaaa agtcaacaat aaaatgaatc ccattgtgtt |
| 5461 | cttttgaaga tgcctatgta acttttaagc tttttaaatt attttcagaa aaaaaaaaag |
| 5521 | aaaagccctt atcagttttc catcagccca ttgccttttt attttttttt tttaatcctt |
| 5581 | gtgaataaat gttctttagt gttttaggag gaaaaagcaa acctagattt tgataaccca |
| 5641 | gaagacttca gattaataaa gaagctttga aagaagacca tttttcaaaa ttttagtgaa |
| 5701 | gtgtgaatat tttttgtcaa tggctttctc aaagagaatg aaacttttgc accattttca |
| 5761 | gagtttttat agagatgcca aattgatata tttacatgta atggaaacat gaaaaagttt |
| 5821 | tattaaacaa ttgttcatag ctgtgtagac attttaattc agtttccaaa gctctcaaaa |
| 5881 | aatcgtattt ttgaagtacg gagtgatgcg gtttggggcg tggcttacag ttccaacgac |
| 5941 | tcaattgtcc cgatactcag ttctttctac aggtatcagg ttcgtgttaa acgctgtatg |
| 6001 | ttaactatga ctggaattct gtgatatttt ggtaataaat gaagtgggat cattgcgaaa |
| 6061 | aaaaaaaaaa aaaaa |

(SEQ ID NO: 2) GenBank accession number: NM_001419.2 (GI: 38201713), incorporated herein by reference.

Exemplary region or fragments of HuR nucleic acid sequences include bases 771-773 (phosphorylation site), bases 2332-2337 (poly A signal sequence), bases 6034-6039 (poly A signal sequence).

### Human HuB Amino Acid Sequence

| | |
|---|---|
| 1 | metqlsngpt cnntangptt innncsspvd sgntedsktn livnylpqnm tgeelksifg |
| 61 | sigeiesckl vrdkitgqsl gygfvnyidp kdaekaintl nglrlqtkti kvsyarpssa |
| 121 | sirdanlyvs gipktmtgke legifsqygr iitsrilvdq vtgisrgvgf irfdkrieae |
| 181 | eaikglngqk ppgatepitv kfannpsqkt nqailsqlyq spnrrypgpl aqqaqrfrld |
| 241 | nllnmaygvk rfspmtidgm tslaginipg hpgtgwcifv ynlapdades ilwqmfgpfg |
| 301 | avtnvkvird fntnkckgfg fvtmtnydea amaiaslngy rlgdrvlqvs fktnkthka |

(SEQ ID NO: 3) GenBank Accession NP_004423.2 (GI: 115511032), incorporated herein by reference.

Exemplary regions or fragments of HuB include residues 38-115 (RNA recognition motif 1), 40-115 (RNA binding site), 120-209 (RNA recognition motif 2), 126-203 (putative RNA binding site), 239-251 (splicing variant), and domain 276-354.

Human HuB nucleotide sequence. The start and stop codons of the coding sequence are bold and underlined.

| | |
|---|---|
| 1 | caataggagg gtagtctctc cgtcttttta aactcttttt taagtttccc ctcccctttc |
| 61 | atattttttt tcgccatttc ttttagcatt ggactttggg gtcgaaagcg tttcttttta |
| 121 | tttgcttctt ttaagccgag cacagtttag gtttcgtgct gtcttaagag aactatccag |
| 181 | cagcttcttg ctcatcctta ttgggagaac tgcaccgtta ctttaaaaac acacatacac |
| 241 | aaaaacctta agggagaaag caggtaattg ctgccatgga aacacaactg tctaatgggc |
| 301 | caacttgcaa taacacagcc aatggtccaa ccaccataaa caacaactgt tcgtcaccag |
| 361 | ttgactctgg gaacacagaa gacagcaaga ccaacttaat agtcaactac cttcctcaga |
| 421 | acatgacaca ggaggaacta aagagtctct ttgggagcat tggtgaaata gagtcctgta |
| 481 | agcttgtaag agacaaaata acagggcaga gcttgggata tggctttgtg aactacattg |
| 541 | accccaagga tgcagagaaa gctatcaaca ccctgaatgg attgagactt caaaccaaaa |
| 601 | caataaaagt ttcctatgct cgcccaagtt cagcttctat cagagatgca aatttatatg |
| 661 | tcagcggact tccaaaaaca atgacccaga aggagttgga acagcttttt tcacaatatg |
| 721 | gacgcattat tacttctcgt attcttgtcg accaggtcac tggcatatca aggggtgtag |
| 781 | ggtttattcg atttgacaag cgaattgagg cagaagaagc tatcaaaggc ctaaatggcc |
| 841 | agaaacctcc cggtgccacg gagccaatca ctgtaaagtt tgctaataac ccaagccaaa |
| 901 | aaaccaatca ggccatcctt tcccagctgt accagtctcc aaacagaagg tatccaggac |
| 961 | cgctagctca gcaggcacag cgttttaggt tggacaatct gctcaatatg gcttatggag |
| 1021 | taaagaggtt ttctccaatg accattgacg gaatgaccag tttggctgga attaatatcc |
| 1081 | ctgggcaccc tggaacaggg tggtgtatat ttgtgtacaa cctggctcct gacgcagatg |
| 1141 | agagtatcct gtggcaaatg tttgggcctt ttggagctgt caccaatgtg aaggtcatcc |
| 1201 | gtgactttaa caccaataaa tgcaaaggtt ttggatttgt gactatgaca aactatgatg |
| 1261 | aggctgccat ggcgatagct agcctcaatg gataccgtct gggagacaga gtactgcagg |
| 1321 | tctcctttaa gacaaacaaa acgcacaaag cctaatgagc tcttgtcctc agtccattta |
| 1381 | tatatgaaaa ctatacaaca aaggcaagtt aagagaaact ttatacatta gtaaatgtct |
| 1441 | ttgtaagtca gtgttgagat ggggataaaa tgactactta gcatcctaag aaatatgtga |
| 1501 | gattttttat tgctagtatt tgaattaaaa cttcttaaat atcttttatg tttgaatatg |
| 1561 | gacaagaggt acagggtttt tacctgtcac attgcattct attgccttct ttgaagaagg |
| 1621 | tggacctttt aaagtgtttc agctaaggga agacatttct tttcttttta cataactgcc |
| 1681 | ttgaacctgt gagtaaatat tgaggctttg tgttgtaatt cttcagttgg ttgtgtcttt |
| 1741 | tttttccccc ctttttttcc tttttctgat tagctttgtg tttggtttac atttaaagca |
| 1801 | ttgctgttat gtctgtttaa gaaaagtatt ttgaagttta catttttatt tatgaagttt |
| 1861 | aaaacagtat ttattttgta attatgattt gggttgggga agggggggct acattataaa |
| 1921 | cgcttattgt aagaatactg gagaactttt cgtaaagcag taccttgcca aagagataag |
| 1981 | agcctctttg atgtgggttt aaaaaaagca tctattttta taaaaaagaa aatttggaga |
| 2041 | aactttttac tggtcctgga acaaatattt tgacttgaat actttgagaa atctcttcat |
| 2101 | atgacaccta gtgagctttt aaaatttacc aggaaatttg cagcggttgg aaaatttaga |
| 2161 | aagatttatg gtgtagaaaa tacttttgag atctttgtat gaaaggagta gaatcaatgg |
| 2221 | ggggaaacac tgctggtttc atttttgtaa tcaccagtgg agcgtctgat catcctggtt |
| 2281 | attatgtgat aggtggctca cattgatttg tgattttgaa acaaataaaa aaaatttaca |
| 2341 | aaagaatata taagagcagg caagaaattt aaattaccga gagatggggg aaaaaatctg |
| 2401 | ttcttcctaa agaaatccct tcagatagag ctcatggtgt ttagtgatgt acttgcagta |
| 2461 | ttgtttgaag aattgttttg tcttaaggaa aaaagacgtt gcacatgatt tgtactgcag |
| 2521 | caaatcagca aaagtgatct gagttggata tatttgaagg tattttgaaa gttacgttca |
| 2581 | aggctaacac ctgagctttg tgtaatgtaa ataagacctt gtgtttatga acctttcagc |
| 2641 | taatttaatt ttttttccct tacatgccaa gtgatgttca ggttttgaat gtttttgtat |
| 2701 | cagttttttc ctttgtaaat ggcattaaca ttgttacttg aggtcttgct taatcacttt |
| 2761 | tgttgtcctg aggacttgaa tttacagtgc atcagatttg ttgcaaattt tgtctgtaga |
| 2821 | tagtctagct tcagctgttt atggtgatgc tacattttcg tttataaata tgtttgtggt |
| 2881 | ataaaaaaat gagtataacc ataggttttg aacaaatttc cttacatttt tcatacaaaa |
| 2941 | atcataaata tctgtatgct attgaaattt aactttgtat gatgcttaaa aaccactatt |
| 3001 | tggggaaata ataaaataag tctttaccat gtatgaaaga aattttaaaa aatacaaaat |
| 3061 | attttctgat tagcatctag cttataataa attttcaaaa aagctgaagg caaaaatgcc |
| 3121 | ttcatcagga tgcactgaga actatatagt tacgtcctgc tttttgtata aactgagatg |
| 3181 | ctcacatgct tccccttaga acaggcaatg tgctatgcat aacatagttg tacattatct |
| 3241 | ttgcggttgc tttgagtttt attttttatt atttaaaatt gtagttataa aatttttcag |
| 3301 | tatagtacag tacatatact gtgaggcgcg tgctaaagtg aataagcgag ttttcatgct |
| 3361 | gacccactca atgctattca gaaatcaatt ggcttagcac tttctcatat ccttaggtgc |
| 3421 | atttagattg ccagagttaa ccttctgcgt ttaaaaaaag aaaaacacta aaaaataaaa |
| 3481 | tacatgtata tacttaaaaa aaaataataa ggtttccctc aagggaaaac agcagctaca |
| 3541 | tgcttctttc ctatactact gtagcaaacc aaggcattga tgagagggca tgcaaattgt |
| 3601 | gcttcacttt acagtgtttt atcagagcac ttaataaaat gtaaggctgg tatttatttg |
| 3661 | aagttgtaca gtatgactta attcacatct gttggaatag aaaatatatt ctgttgagta |
| 3721 | tttaagaggc tgtacatgtt ttcttttgtg tttggattct ttgtactttt tcatgttcag |
| 3781 | tacatcaata aacaaagttg aagggaaaaa aaaa |

(SEQ ID NO: 4) GenBank accession number: NM_004432.3 (GI: 283945527), incorporated herein by reference.

Exemplary regions or fragments of HuB nucleic acid sequence include bases 936-938 (phosphorylation site), and 3787-3792 (regulator sequence, polyA signal sequence).

### Detection of Circulating HU Protein for Assessment of Heart Failure and Arrhythmic Risk Stratification

HU family proteins play an important role in regulation SCN5A expression by stabilizing SCN5A mRNA. HuR mRNA was decreased 38.9% (P=0.034) in peripheral white blood cells of heart failure patients with an ICD and evidence of appropriate event-driven therapy. The decrease of HuR mRNA correlated with a reduction of wild-type SCN5A mRNA expression in these patients. The results demonstrated an additional layer of SCN5A posttranscriptional regulation in addition to alternative splicing that contributes to sodium channel downregulation in heart failure. These results also indicated that circulating HU protein assessment is useful in arrhythmic risk stratification and that therapy to induce HU protein elevation reduces arrhythmic risk in heart failure.

Circulating HU proteins, such as HuR, are detected using methods known in the art, e.g., by binding to HuR specific antibodies. Such antibodies are publically available, e.g., Santa Cruz Biotechnology anti-HuR antibody (catalog number sc20694 and sc-5261) and Life Technologies Corporation HuR antibody (catalog number 39-0600). For example, a HuR antibody binds to the HuR protein such as an RRM 1, 2, or 3 domains. Binding is detected using known methods such as an ELISA assay or Western blot techniques. Alternatively, a nucleic acid based detection approach is used, e.g., to detect a HuR gene transcript (see nucleotide sequence above) using methods known in the art such as PCR, or Northern blot techniques.

Human Heart Failure is Associated with Sodium Channel mRNA Degradation Mediated by Reduced Level of HU Proteins

Human heart failure (HF) has been associated with reduced cardiac sodium channel current, in part because of a reduction in SCN5A mRNA abundance. Reduced SCN5A contributes to arrhythmic risk in heart failure. The reduction in cardiac SCN5A mRNA abundance is reflected in circulating white cells that also express SCN5A.

Studies were carried out to evaluate whether reduced mRNA stability contributes to reduced SCN5A mRNA abundance in heart failure. The SCN5A mRNA 3′-untranslated region (UTR) contains two sets of AU-rich elements (ARE), which mediate ARE-mediated mRNA decay. HU proteins, members of the ARE-binding protein family, were overexpressed in human cardiomyocytes and were found to increase wild-type SCN5A mRNA level significantly. Wild-type SCN5A mRNA increased 46.3% (P=0.004) and 60.7% (P=0.008) with HuR and HuB overexpression, respectively. These data indicated that HU family proteins play an important role in regulation SCN5A expression by stabilizing SCN5A mRNA.

The methods, e.g., blood test, and kits described herein are used to predict arrhythmic risk and to determine the need for an implanted defibrillator as well as to determine heart failure severity.

### SCN5A (Sodium Channel, Voltage Gated, Type V Alpha Protein)

Cardiac voltage-gated Na⁺ (Nay) channels consist of a heteromeric assembly of pore-forming a subunit and auxiliary β subunits that modulate channel functions. Nav1.5 (SCN5A) is the major Nay a subunit expressed in the mammalian myocardium, whereas multiple Nay β subunits have been described in cardiomyocytes. Voltage-gated Na⁺ channels play a critical role in the membrane excitability of cardiomyocytes by generating the rapid upstroke of the action potential. Additionally, Nay channels govern the impulse conduction velocity in the myocardium. Abnormal cardiac Na⁺ channel function has been associated in hereditary cardiac diseases such as long QT syndrome (LQTS), Brugada syndrome, and progressive cardiac conduction defect, as well as acquired cardiac conditions including myocardial ischemia and heart failure.

The method of identifying a subject at risk for arrhythmias or heart failure comprises the step of determining a level of a full length transcript of SCN5A gene or of a splice variant of the SCN5A gene. Splice variants of the SCN5A gene are further described in the art, See, e.g., PCT application 2012/0129179, and PCT application PCT/US2012/20564, incorporated herein by reference.

The method comprises determining a level of a full length transcript of SCN5A gene, and a decreased level of the full length transcript of the SCN5A gene indicates an increased risk for arrhythmia or heart failure. In exemplary aspects, the method comprises determining a level of a splice variant of the SCN5A gene, and an increased level of the splice variant indicates an increased risk for arrhythmia or heart failure. In specific aspects, the splice variant of the SCN5A gene is a splice variant produced from alternative splicing within Exon 28 of the SCN5A gene. In specific aspects, the splice variant is a SCN5A Exon 28 B splice variant (a.k.a., E28B SEQ ID NO: 5), a SCN5A Exon 28 C splice variant (a.k.a., E28C SEQ ID NO: 6), or a SCN5A Exon 28 D splice variant (a.k.a., E28D SEQ ID NO7).

The level may be an expression level of a full length transcript of SCN5A gene or of a splice variant of the SCN5A gene. Suitable methods of determining expression levels of transcripts of a gene are include direct methods of determining levels of transcripts (e.g., quantitative PCR) and indirect methods of determining levels of transcripts (e.g., Western blotting for translated protein or peptide products of the transcripts). The level may be an activity level of a full-length transcript of the SCN5A gene that is determined via measurement, e.g., measurement of the sodium current.

### SCN5A Splice Variant Exon 28 B (E28B)

| (SEQ ID NO: 5) | |
|---|---|
| ggagccctcc tagtgagtat gaagtgatat ctcactgagg ttttggtttg caaaagcaaa | 60 |
| tgactgatga ctaacgatgc aggacatctt tccatgtgca tgttggtcat ttatatatct | 120 |
| tccttggaga aatctctatt cagatcctta gctcattttt aattgggtta tttctcttct | 180 |
| tcttgttgag ttgtaagagt tctttacata ttctggatca cagtctctta tcagatatat | 240 |
| gatttaaaaa tattttctcc tagtctgtga gttttttcat ttcctagtgg tgtccattaa | 300 |
| agcacaaaag ttttacatgt t | 321 |

### SCN5A Splice Variant Exon 28 C (E28C)

| (SEQ ID NO: 6) | |
|---|---|
| gaactgcaca atgaccagca ggaggggaga agagagtagg aaaaaggagg gaaggacaga | 60 |
| catcaagtgc cagatgttgt ctgaactaat cgagcacttc tcaccaaact tcatgtataa | 120 |
| ataaaataca tatttttaaa acaaaccaat aaatggctta catg | 164 |

### SCN5A Splice Variant Exon 28 D (E28D)

| (SEQ ID NO: 7) | |
|---|---|
| ggcactgtgc tctcggacat catccagaag tacttcttct ccccgacgct cttccgagtc | 60 |
| atccgcctgg cccgaatagg ccgcatcctc agactgatcc gaggggccaa gggg | 114 |

### SCN5A Amino Acid Sequence

| | |
|---|---|
| 1 | manfllprgt ssfrrftres laaiekrmae kgargsttlq esreglpeee aprpqldlqa |
| 61 | skklpdlygn ppgeligepl edldpfystq ktfivinkgk tifrfsatna lyvlspfhpi |
| 121 | rraavkilvh slfnmlimct iltncvfmaq hdpppwtkyv eytftaiytf eslvkilarg |
| 181 | fclhaftflr dpwnwldfsv iimayttefv dlgnvsalrt frvlralkti svisglktiv |
| 241 | galigsvkkl advmvltvfc lsvfaliglq lfmgnlrhkc vrnftalngt ngsveadglv |
| 301 | wesldlylsd penyllkngt sdvllcgnss dagtcpegyr clkagenpdh gytsfdsfaw |
| 361 | aflalfrlmt qdcwerlyqg tlrsagkiym iffmlviflg sfylvnlila vvamayeeqn |
| 421 | qatiaeteek ekrfqeamem lkkehealti rgvdtvsrss lemsplapvn sherrskrrk |
| 481 | rmssgteecg edrlpksdse dgpramnhls ltrglsrtsm kprssrgsif tfrrrdlgse |
| 541 | adfaddenst ageseshhts llvpwplrrt sagggpspgt sapghalhgk knstvdcngv |
| 601 | vsllgagdpe atspgshllr pvmlehppdt ttpseepggp gmltsqapcv dgfeepgarq |
| 661 | ralsaysvlt saleeleesr hkcppcwnrl aqryliwecc plwmsikqgv klvvmdpftd |
| 721 | ltitmcivin tlfmalehyn mtsefeemlq vgnlvftgif taemtfkiia ldpyyyfqqg |
| 781 | wnifdsiivi lslmelglsr msnlsvlrsf rllrvfklak swptlntlik iignsvgalg |
| 841 | nitivlaiiv fifavvgmql fgknyselrd sdsgllprwh mmdffhafli ifrilcgewi |
| 901 | etmwdcmevs ggslcllvfl lvmvignlvv lnlflallls sfsadnitap dedremnnlq |
| 961 | lalariqrgl rfvkrttwdf ccgllrqrpq kpaalaaqgq lpsciatpys ppppetekvp |
| 1021 | ptrketrfee geqpgqgtpg dpepvcvpia vaesdtddqe edeenslgte eesskqqesq |
| 1081 | pvsggpeapp dsrtwsqvsa tasseaeasa sqadwrqqwk aepqapgcge tpedscsegs |
| 1141 | tadmtntael leqipdlgqd vkdpedcfte gcvrrcpcca vdttqapgkv wwrlrktcyh |
| 1201 | ivehswfetf iifmillssg alafediyle erktikvlle yadkmftyvf vlemllkwva |
| 1261 | ygfkkyftna wcwldflivd vslvslvant lgfaemgpik slrtlralrp lralsrfegm |
| 1321 | rvvvnalvga ipsimnvllv clifwlifsi mgvnlfagkf grcinqtegd 1pinytivnn |
| 1381 | ksqceslnit gelywtkvkv nfdnvgagyl allqvatfkg wmdimyaavd srgyeeqpqw |
| 1441 | eynlymyiyf vifiifgsff tlnlfigvii dnfnqqkkkl ggqdifmtee qkkyynamkk |
| 1501 | lgskkpqkpi prpinkyqgf ifdivtkqaf dvtimflicl nmvtmmvetd dqspekinil |
| 1561 | akinllfvai ftgecivkla alrhyyftns wnifdfvvvi lsivgtvlsd iiqkyffspt |
| 1621 | lfrvirlari grilrlirga kgirtllfal mmslpalfni glllflvmfi ysifgmanfa |
| 1681 | yvkweagidd mfnfqtfans mlclfqitts agwdgllspi lntgppycdp tlpnsngsrg |
| 1741 | dcgspavgil ffttyiiisf livvnmyiai ilenfsvate esteplsedd fdmfyeiwek |
| 1801 | fdpeatqfie ysvlsdfada lseplriakp ngislinmdl pmvsgdrihc mdilfaftkr |
| 1861 | vlgesgemda lkiqmeekfm aanpskisye pitttlrrkh eevsamviqr afrrhllqrs |
| 1921 | lkhasflfrq qagsglseed aperegliay vmsenfsrpl gppssssiss tsfppsydsv |
| 1981 | tratsdnlqv rgsdyshsed ladfppspdr dresiv |

(SEQ ID NO: 12) GenBank Accession NP_932173.1 (GI: 37622907), incorporated herein by reference.

Exemplary regions or fragments of SCN5A include residues 159-412 (ion transport region), 159-178 (transmembrane region), 842-862 (transmembrane region), and 1201-1224 (sodium ion transport-associated region).

SCN5A nucleotide sequence. The start and stop codons of the coding sequence are bold and underlined.

| | |
|---|---|
| 1 | agacggcggc ggcgcccgta ggatgcaggg atcgctcccc cggggccgct gagcctgcgc |
| 61 | ccagtgcccc gagccccgcg ccgagccgag tccgcgccaa gcagcagccg cccaccccgg |
| 121 | ggcccggccg ggggaccagc agcttcccca caggcaacgt gaggagagcc tgtgcccaga |
| 181 | agcaggatga gaagatggca aacttcctat tacctcgggg caccagcagc ttccgcaggt |
| 241 | tcacacggga gtccctggca gccatcgaga agcgcatggc agagaagcaa gcccgcggct |
| 301 | caaccacctt gcaggagagc cgagaggggc tgcccgagga ggaggctccc cggccccagc |
| 361 | tggacctgca ggcctccaaa aagctgccag atctctatgg caatccaccc caagagctca |
| 421 | tcggagagcc cctggaggac ctggacccct tctatagcac ccaaaagact ttcatcgtac |
| 481 | tgaataaagg caagaccatc ttccggttca gtgccaccaa cgccttgtat gtcctcagtc |
| 541 | ccttccaccc catccggaga gcggctgtga agattctggt tcactcgctc ttcaacatgc |
| 601 | tcatcatgtg caccatcctc accaactgcg tgttcatggc ccagcacgac cctccaccct |
| 661 | ggaccaagta tgtcgagtac accttcaccg ccatttacac ctttgagtct ctggtcaaga |
| 721 | ttctggctcg aggcttctgc ctgcacgcgt tcactttcct tcgggaccca tggaactggc |
| 781 | tggactttag tgtgattatc atggcataca caactgaatt tgtggacctg ggcaatgtct |
| 841 | cagccttacg caccttccga gtcctccggg ccctgaaaac tatatcagtc atttcagggc |
| 901 | tgaagaccat cgtgggggcc ctgatccagt ctgtgaagaa gctggctgat gtgatggtcc |
| 961 | tcacagtctt ctgcctcagc gtctttgccc tcatcggcct gcagctcttc atgggcaacc |
| 1021 | taaggcacaa gtgcgtgcgc aacttcacag cgctcaacgg caccaacggc tccgtggagg |
| 1081 | ccgacggctt ggtctgggaa tccctggacc tttacctcag tgatccagaa aattacctgc |
| 1141 | tcaagaacgg cacctctgat gtgttactgt gtgggaacag ctctgacgct gggacatgtc |
| 1201 | cggagggcta ccggtgccta aaggcaggcg agaaccccga ccacggctac accagcttcg |
| 1261 | attcctttgc ctgggccttt cttgcactct tccgcctgat gacgcaggac tgctgggagc |
| 1321 | gcctctatca gcagaccctc aggtccgcag ggaagatcta catgatcttc ttcatgcttg |
| 1381 | tcatcttcct ggggtccttc tacctggtga acctgatcct ggccgtggtc gcaatggcct |
| 1441 | atgaggagca aaaccaagcc accatcgctg agaccgagga gaaggaaaag cgcttccagg |
| 1501 | aggccatgga aatgctcaag aaagaacacg aggccctcac catcaggggt gtggataccg |
| 1561 | tgtcccgtag ctccttggag atgtcccctt tggccccagt aaacagccat gagagaagaa |
| 1621 | gcaagaggag aaaacggatg tcttcaggaa ctgaggagtg tggggaggac aggctcccca |
| 1681 | agtctgactc agaagatggt cccagagcaa tgaatcatct cagcctcacc cgtggcctca |
| 1741 | gcaggacttc tatgaagcca cgttccagcc gcgggagcat tttcaccttt cgcaggcgag |
| 1801 | acctgggttc tgaagcagat tttgcagatg atgaaaacag cacagcgggg gagagcgaga |
| 1861 | gccaccacac atcactgctg gtgccctggc ccctgcgccg gaccagtgcc cagggacagc |
| 1921 | ccagtcccgg aacctcggct cctggccacg ccctccatgg caaaaagaac agcactgtgg |
| 1981 | actgcaatgg ggtggtctca ttactggggg caggcgaccc agaggccaca tccccaggaa |
| 2041 | gccacctcct ccgccctgtg atgctagagc acccgccaga cacgaccacg ccatcggagg |
| 2101 | agccaggcgg gccccagatg ctgacctccc aggctccgtg tgtagatggc ttcgaggagc |
| 2161 | caggagcacg gcagcgggcc ctcagcgcag tcagcgtcct caccagcgca ctggaagagt |
| 2221 | tagaggagtc tcgccacaag tgtccaccat gctggaaccg tctcgcccag cgctacctga |
| 2281 | tctgggagtg ctgcccgctg tggatgtcca tcaagcaggg agtgaagttg gtggtcatgg |
| 2341 | acccgtttac tgacctcacc atcactatgt gcatcgtact caacacactc ttcatggcgc |
| 2401 | tggagcacta caacatgaca agtgaattcg aggagatgct gcaggtcgga aacctggtct |
| 2461 | tcacagggat tttcacagca gagatgacct tcaagatcat tgccctcgac ccctactact |
| 2521 | acttccaaca gggctggaac atcttcgaca gcatcatcgt catccttagc ctcatggagc |
| 2581 | tgggcctgtc ccgcatgagc aacttgtcgg tgctgcgctc cttccgcctg ctgcgggtct |
| 2641 | tcaagctggc caaatcatgg cccaccctga acacactcat caagatcatc gggaactcag |
| 2701 | tgggggcact ggggaacctg acactggtgc tagccatcat cgtgttcatc tttgctgtgg |
| 2761 | tgggcatgca gctctttggc aagaactact cggagctgag ggacagcgac tcaggcctgc |
| 2821 | tgcctcgctg gcacatgatg gacttctttc atgccttcct catcatcttc cgcatcctct |
| 2881 | gtggagagtg gatcgagacc atgtgggact gcatggaggt gtcggggcag tcattatgcc |
| 2941 | tgctggtctt cttgcttgtt atggtcattg gcaaccttgt ggtcctgaat ctcttcctgg |
| 3001 | ccttgctgct cagctccttc agtgcagaca acctcacagc ccctgatgag gacagagaga |
| 3061 | tgaacaacct ccagctggcc ctggcccgca tccagagggg cctgcgcttt gtcaagcgga |
| 3121 | ccacctggga tttctgctgt ggtctcctgc ggcagcggcc tcagaagccc gcagcccttg |
| 3181 | ccgcccaggg ccagctgccc agctgcattg ccacccccta ctccccgcca cccccagaga |
| 3241 | cggagaaggt gcctcccacc cgcaaggaaa cacggtttga ggaaggcgag caaccaggcc |
| 3301 | agggcacccc cggggatcca gagcccgtgt gtgtgcccat cgctgtggcc gagtcagaca |
| 3361 | cagatgacca agaagaagat gaggagaaca gcctgggcac ggaggaggag tccagcaagc |
| 3421 | agcaggaatc ccagcctgtg tccggtggcc cagaggcccc tccggattcc aggacctgga |
| 3481 | gccaggtgtc agcgactgcc tcctctgagg ccgaggccag tgcatctcag gccgactggc |
| 3541 | ggcagcagtg gaaagcggaa ccccaggccc cagggtgcgg tgagacccca gaggacagtt |
| 3601 | gctccgaggg cagcacagca gacatgacca acaccgctga gctcctggag cagatccctg |
| 3661 | acctcggcca ggatgtcaag gacccagagg actgcttcac tgaaggctgt gtccggcgct |
| 3721 | gtccctgctg tgcggtggac accacacagg ccccagggaa ggtctggtgg cggttgcgca |
| 3781 | agacctgcta ccacatcgtg gagcacagct ggttcgagac attcatcatc ttcatgatcc |
| 3841 | tactcagcag tggagcgctg gccttcgagg acatctacct agaggagcgg aagaccatca |
| 3901 | aggttctgct tgagtatgcc gacaagatgt tcacatatgt cttcgtgctg gagatgctgc |
| 3961 | tcaagtgggt ggcctacggc ttcaagaagt acttcaccaa tgcctggtgc tggctcgact |
| 4021 | tcctcatcgt agacgtctct ctggtcagcc tggtggccaa caccctgggc tttgccgaga |
| 4081 | tgggccccat caagtcactg cggacgctgc gtgcactccg tcctctgaga gctctgtcac |
| 4141 | gatttgaggg catgagggtg gtggtcaatg ccctggtggg cgccatcccg tccatcatga |
| 4201 | acgtcctcct cgtctgcctc atcttctggc tcatcttcag catcatgggc gtgaacctct |
| 4261 | ttgcggggaa gtttgggagg tgcatcaacc agacagaggg agacttgcct ttgaactaca |
| 4321 | ccatcgtgaa caacaagagc cagtgtgagt ccttgaactt gaccggagaa ttgtactgga |
| 4381 | ccaaggtgaa agtcaacttt gacaacgtgg gggccgggta cctggccctt ctgcaggtgg |
| 4441 | caacatttaa aggctggatg gacattatgt atgcagctgt ggactccagg gggtatgaag |
| 4501 | agcagcctca gtgggaatac aacctctaca tgtacatcta ttttgtcatt ttcatcatct |
| 4561 | ttgggtcttt cttcaccctg aacctcttta ttggtgtcat cattgacaac ttcaaccaac |
| 4621 | agaagaaaaa gttagggggc caggacatct tcatgacaga ggagcagaag aagtactaca |
| 4681 | atgccatgaa gaagctgggc tccaagaagc cccagaagcc catcccacgg cccctgaaca |
| 4741 | agtaccaggg cttcatattc gacattgtga ccaagcaggc ctttgacgtc accatcatgt |
| 4801 | ttctgatctg cttgaatatg gtgaccatga tggtggagac agatgaccaa agtcctgaga |
| 4861 | aaatcaacat cttggccaag atcaacctgc tctttgtggc catcttcaca ggcgagtgta |
| 4921 | ttgtcaagct ggctgccctg cgccactact acttcaccaa cagctggaat atcttcgact |
| 4981 | tcgtggttgt catcctctcc atcgtgggca ctgtgctctc ggacatcatc cagaagtact |
| 5041 | tcttctcccc gacgctcttc cgagtcatcc gcctggcccg aataggccgc atcctcagac |
| 5101 | tgatccgagg ggccaagggg atccgcacgc tgctctttgc cctcatgatg tccctgcctg |
| 5161 | ccctcttcaa catcgggctg ctgctcttcc tcgtcatgtt catctactcc atctttggca |
| 5221 | tggccaactt cgcttatgtc aagtgggagg ctggcatcga cgacatgttc aacttccaga |
| 5281 | ccttcgccaa cagcatgctg tgcctcttcc agatcaccac gtcggccggc tgggatggcc |
| 5341 | tcctcagccc catcctcaac actgggccgc cctactgcga ccccactctg cccaacagca |
| 5401 | atggctctcg gggggactgc gggagcccag ccgtgggcat cctcttcttc accacctaca |
| 5461 | tcatcatctc cttcctcatc gtggtcaaca tgtacattgc catcatcctg gagaacttca |
| 5521 | gcgtggccac ggaggagagc accgagcccc tgagtgagga cgacttcgat atgttctatg |
| 5581 | agatctggga gaaatttgac ccagaggcca ctcagtttat tgagtattcg gtcctgtctg |
| 5641 | actttgccga tgccctgtct gagccactcc gtatcgccaa gcccaaccag ataagcctca |
| 5701 | tcaacatgga cctgcccatg gtgagtgggg accgcatcca ttgcatggac attctctttg |
| 5761 | ccttcaccaa aagggtcctg ggggagtctg gggagatgga cgccctgaag atccagatgg |
| 5821 | aggagaagtt catggcagcc aacccatcca agatctccta cgagcccatc accaccacac |
| 5881 | tccggcgcaa gcacgaagag gtgtcggcca tggttatcca gagagccttc cgcaggcacc |
| 5941 | tgctgcaacg ctctttgaag catgcctcct tcctcttccg tcagcaggcg ggcagcggcc |
| 6001 | tctccgaaga ggatgcccct gagcgagagg gcctcatcgc ctacgtgatg agtgagaact |
| 6061 | tctcccgacc ccttggccca ccctccagct cctccatctc ctccacttcc ttcccaccct |
| 6121 | cctatgacag tgtcactaga gccaccagcg ataacctcca ggtgcggggg tctgactaca |
| 6181 | gccacagtga agatctcgcc gacttccccc cttctccgga cagggaccgt gagtccatcg |
| 6241 | tgtgagcctc ggcctggctg gccaggacac actgaaaagc agcctttttc accatggcaa |
| 6301 | acctaaatgc agtcagtcac aaaccagcct ggggccttcc tggctttggg agtaagaaat |
| 6361 | gggcctcagc cccgcggatc aaccaggcag agttctgtgg cgccgcgtgg acagccggag |
| 6421 | cagttggcct gtgcttggag gcctcagata gacctgtgac ctggtctggt caggcaatgc |
| 6481 | cctgcggctc tggaaagcaa cttcatccca gctgctgagg cgaaatataa aactgagact |
| 6541 | gtatatgttg tgaatgggct ttcataaatt tattatattt gatatttttt tacttgagca |
| 6601 | aagaactaag gatttttcca tggacatggg cagcaattca cgctgtctct tcttaaccct |
| 6661 | gaacaagagt gtctatggag cagccggaag tctgttctca aagcagaagt ggaatccagt |
| 6721 | gtggctccca caggtcttca ctgcccaggg gtcgaatggg gtccccctcc cacttgacct |
| 6781 | gagatgctgg gagggctgaa cccccactca cacaagcaca cacacacagt cctcacacac |
| 6841 | ggaggccaga cacaggccgt gggacccagg ctcccagcct aagggagaca ggcctttccc |
| 6901 | tgccggcccc ccaaggatgg ggttcttgtc cacggggctc actctggccc cctattgtct |
| 6961 | ccaaggtccc attttccccc tgtgttttca cgcaggtcat attgtcagtc ctacaaaaat |
| 7021 | aaaaggcttc cagaggagag tggcctgggt cccagggctg gccctaggca ctgatagttg |
| 7081 | ccttttcttc ccctcctgta agagtattaa caaaaccaaa ggacacaagg gtgcaagccc |
| 7141 | cattcacggc ctggcatgca gcttgtcctt gctcctggaa cctggcaggc cctgcccagc |
| 7201 | cagccatcgg aagagagggc tgagccatgg gggtttgggg ctaagaagtt caccagccct |
| 7261 | gagccatggc ggcccctcag cctgcctgaa gagaggaaac tggcgatctc ccagggctct |
| 7321 | ctggaccata cgcggaggag ttttctgtgt ggtctccagc tcctctccag acacagagac |
| 7381 | atgggagtgg ggagcggagc ttggccctgc gccctgtgca gggaaaggga tggtcaggcc |
| 7441 | cagttctcgt gcccttagag gggaatgaac catggcacct ttgagagagg gggcactgtg |
| 7501 | gtcaggccca gcctctctgg ctcagcccgg gatcctgatg gcacccacac agaggacctc |
| 7561 | tttggggcaa gatccaggtg gtcccatagg tcttgtgaaa aggctttttc agggaaaaat |
| 7621 | attttactag tccaatcacc cccaggacct cttcagctgc tgacaatcct atttagcata |
| 7681 | tgcaaatctt ttaacataga gaactgtcac cctgaggtaa cagggtcaac tggcgaagcc |
| 7741 | tgagcaggca ggggcttggc tgccccattc cagctctccc atggagcccc tccaccgggc |
| 7801 | gcatgcctcc caggccacct cagtctcacc tgccggctct gggctggctg ctcctaacct |
| 7861 | acctcgccga gctgtcggag ggctggacat ttgtggcagt gctgaagggg gcattgccgg |
| 7921 | cgagtaaagt attatgtttc ttcttgtcac cccagttccc ttggtggcaa ccccagaccc |
| 7981 | aacccatgcc cctgacagat ctagttctct tctcctgtgt tccctttgag tccagtgtgg |
| 8041 | gacacggttt aactgtccca gcgacatttc tccaagtgga aatcctattt ttgtagatct |
| 8101 | ccatgctttg ctctcaaggc ttggagaggt atgtgcccct cctgggtgct caccgcctgc |
| 8161 | tacacaggca ggaatgcggt tgggaggcag gtcgggctgc cagcccagct ggccggaagg |
| 8221 | agactgtggt ttttgtgtgt gtggacagcc cgggagcttt gagacaggtg cctggggctg |
| 8281 | gctgcagacg gtgtggttgg gggtgggagg tgagctagac ccaaccctta gcttttagcc |
| 8341 | tggctgtcac ctttttaatt tccagaactg cacaatgacc agcaggaggg aaggacagac |
| 8401 | atcaagtgcc agatgttgtc tgaactaatc gagcacttct caccaaactt catgtataaa |
| 8461 | taaaatacat atttttaaaa caaaccaata aatggcttac atga |

(SEQ ID NO: 13) GenBank Accession NM_198056.2 (GI: 124518659), incorporated herein by reference.

Exemplary regions or fragments of SCN5A include residues 95-1022 (transmembrane region), 1563-1565 (phosphorylation site), 1731-1733 (methylation site), and 5172-5240 (transmembrane region).

### MEF2C (Myocyte-Specific Enhancer Factor 2C)

MEF2C, also known as MADS box transcription enhancer factor 2, polypeptide c is involved in cardiac morphogenesis, myogenesis and vascular development. The myocyte enhancer factor-2 (MEF-2) family of transcription factors associate with co-repressors or co-activators to regulate development and function of T cells, neuronal cells, and muscle cells. Four family members arise from alternatively spliced transcripts, termed MEF2A, -2B, -2C, and -2D, These members bind as homo- and heterodimers to the MEF2 site in the promoter region of affected genes. Differential regulation in the expression of the four transcripts implies functional distinction for each during embryogenesis and development. The process of differentiation from mesodermal precursor cells to myoblasts has led to the discovery of a variety of tissue-specific factors that regulate muscle gene expression. The myogenic basic helix-loop-helix proteins, including, MyoD, myooenin. Myf-5, and MRF4, are one class of identified factors. A second family of DNA binding regulatory proteins is the myocyte-specific enhancer factor-2 (MEF-2) family, Each of these proteins binds to the MEF-2 target DNA sequence present in the regulatory regions of many muscle-specific genes. In adult tissues, Mef2 proteins regulate the seers-response during cardiac hypertrophy and tissue remodeling in cardiac and skeletal muscle.

### Human MEF2C Amino Acid Sequence

| | |
|---|---|
| 1 | mgrkkiqitr imdernrqvt ftkrkfglmk kayelsvlcd ceialiifns tnklfqyast |
| 61 | dmdkvllkyt eynephesrt nsdivetlrk kglngcdspd pdaddsvghs pesedkyrki |
| 121 | nedidlmisr qrlcavpppn fempvsipvs shnslvysnp vsslgnpnll plahpslqrn |
| 181 | smspgvthrp psagntgglm ggdltsgagt sagngygnpr nspgllvspg nlnknmqaks |
| 241 | pppmnlgmnn rkpdlrvlip pgskntmpsv sedvdlllnq rinnsgsags latpvvsvat |
| 301 | ptlpgqgmgg ypsaisttyg teyslssadl sslsgfntas alhlgsvtgw qqqhlhnmpp |
| 361 | salsqlgact sthlsgssnl slpstqslni ksepvspprd rtttpsrypq htrheagrsp |
| 421 | vdslsscsss ydgsdredhr nefhspiglt rpspderesp svkrmrlseg wat |

(SEQ ID NO: 8) GenBank Accession NP_001180279.1 (GI: 301069386), incorporated herein by reference.

Exemplary regions or fragments of MEF2C include residues 2-38 (DNA binding site), 3-57 (binding domain), 4-31 (compositionally biased region, lysine rich region), 21-73 (dimerization interface), 87-134 (splicing variant), 107-134 (splicing variant), 110-156 (Holliday junction regulator protein family), 271-278 (splicing variant), and 368-399 (transcription repressor binding site).

Human MEF2C nucleotide sequence. The start and stop codons of the coding sequence are bold and underlined.

| | |
|---|---|
| 1 | aagggggcaa agcctcggtc ttcatagaaa aggagaggag gcaaacgcag cccaaactgg |
| 61 | ggggtttctc ttcaaagcca gctggtctgg ctttattctg caggaatttt tttacctgtc |
| 121 | agggtttgga caacaaagcc ctcagcaggt gctgacgggt acaacttcct ggagaagcag |
| 181 | aaaggcactg gtgccaaaga agagttgcaa actgtgaagt aacttctatg aagagatgaa |
| 241 | gtaaagaacg gaaggcaaat gattgtggca gtaaagaagt gtatgtgcag gaacgaatgc |
| 301 | aggaatttgg gaactgagct gtgcaagtgc tgaagaagga gatttgtttg gaggaaacag |
| 361 | gaaagagaaa gaaaaggaag gaaaaaatac ataatttcag ggacgagaga gagaagaaaa |
| 421 | acggggacta tggggagaaa aaagattcag attacgagga ttatggatga acgtaacaga |
| 481 | caggtgacat ttacaaagag gaaatttggg ttgatgaaga aggcttatga gctgagcgtg |
| 541 | ctgtgtgact gtgagattgc gctgatcatc ttcaacagca ccaacaagct gttccagtat |
| 601 | gccagcaccg acatggacaa agtgcttctc aagtacacgg agtacaacga gccgcatgag |
| 661 | agccggacaa actcagacat cgtggaggca ttgaacaaga aagaaaacaa aggctgtgaa |
| 721 | agccccgatc ccgactcctc ttatgcactc accccacgca ctgaagaaaa atacaaaaaa |
| 781 | attaatgaag aatttgataa tatgatcaag agtcataaaa ttcctgctgt tccacctccc |
| 841 | aacttcgaga tgccagtctc catcccagtg tccagccaca acagtttggt gtacagcaac |
| 901 | cctgtcagct cactgggaaa ccccaaccta ttgccactgg ctcacccttc tctgcagagg |
| 961 | aatagtatgt ctcctggtgt aacacatcga cctccaagtg caggtaacac aggtggtctg |
| 1021 | atgggtggag acctcacgtc tggtgcaggc accagtgcag ggaacgggta tggcaatccc |
| 1081 | cgaaactcac caggtctgct ggtctcacct ggtaacttga acaagaatat gcaagcaaaa |
| 1141 | tctcctcccc caatgaattt aggaatgaat aaccgtaaac cagatctccg agttcttatt |
| 1201 | ccaccaggca gcaagaatac gatgccatca gtgaatcaaa ggataaataa ctcccagtcg |
| 1261 | gctcagtcat tggctacccc agtggtttcc gtagcaactc ctactttacc aggacaagga |
| 1321 | atgggaggat atccatcagc catttcaaca acatatggta ccgagtactc tctgagtagt |
| 1381 | gcagacctgt catctctgtc tgggtttaac accgccagcg ctcttcacct tggttcagta |
| 1441 | actggctggc aacagcaaca cctacataac atgccaccat ctgccctcag tcagttggga |
| 1501 | gcttgcacta gcactcattt atctcagagt tcaaatctct ccctgccttc tactcaaagc |
| 1561 | ctcaacatca agtcagaacc tgtttctcct cctagagacc gtaccaccac cccttcgaga |
| 1621 | tacccacaac acacgcgcca cgaggcgggg agatctcctg ttgacagctt gagcagctgt |
| 1681 | agcagttcgt acgacgggag cgaccgagag gatcaccgga acgaattcca ctcccccatt |
| 1741 | ggactcacca gaccttcgcc ggacgaaagg gaaagtccct cagtcaagcg catgcgactt |
| 1801 | tctgaaggat gggcaacatg atcagattat tacttactag tttttttttt tttcttgcag |
| 1861 | tgtgtgtgtg tgctatacct taatggggaa ggggggtcga tatgcattat atgtgccgtg |
| 1921 | tgtggaaaaa aaaaaagtca ggtactctgt tttgtaaaag tacttttaaa ttgcctcagt |
| 1981 | gatacagtat aaagataaac agaaatgctg agataagctt agcacttgag ttgtacaaca |
| 2041 | gaacacttgt acaaaataga ttttaaggct aacttctttt cactgttgtg ctcctttgca |
| 2101 | aaatgtatgt tacaatagat agtgtcatgt tgcaggttca acgttattta catgtaaata |
| 2161 | gacaaaagga aacatttgcc aaaagcggca gatctttact gaaagagaga gcagctgtta |
| 2221 | tgcaacatat agaaaaatgt atagatgctt ggacagaccc ggtaatgggt ggccattggt |
| 2281 | aaatgttagg aacacaccag gtcacctgac atcccaagaa tgctcacaaa cctgcaggca |
| 2341 | tatcattggc gtatggcact cattaaaaag gatcagagac cattaaaaga ggaccatacc |
| 2401 | tattaaaaaa aaatgtggag ttggagggct aacatattta attaaataaa taaataaatc |
| 2461 | tgggtctgca tctcttatta aataaaaata taaaaatatg tacattacat tttgcttatt |
| 2521 | ttcatataaa aggtaagaca gagtttgcaa agcatttgtg gctttttgta gtttacttaa |
| 2581 | gccaaaatgt gtttttttcc ccttgatagc ttcgctaata ttttaaacag tcctgtaaaa |
| 2641 | aaccaaaaag gactttttgt atagaaagca ctaccctaag ccatgaagaa ctccatgctt |
| 2701 | tgctaaccaa gataactgtt ttctctttgt agaagttttg tttttgaaat gtgtatttct |
| 2761 | aattatataa aatattaaga atcttttaaa aaaatctgtg aaattaacat gcttgtgtat |
| 2821 | agctttctaa tatatataat attatggtaa tagcagaagt tttgttatct taatagcggg |
| 2881 | aggggggtat atttgtgcag ttgcacattt gagtaactat tttctttctg ttttctttta |
| 2941 | ctctgcttac attttataag tttaaggtca gctgtcaaaa ggataacctg tggggttaga |
| 3001 | acatatcaca ttgcaacacc ctaaattgtt tttaatacat tagcaatcta ttgggtcaac |
| 3061 | tgacatccat tgtatatact agtttctttc atgctatttt tattttgttt tttgcatttt |
| 3121 | tatcaaatgc agggcccctt tctgatctca ccatttcacc atgcatcttg gaattcagta |
| 3181 | agtgcatatc ctaacttgcc catattctaa atcatctggt tggttttcag cctagaattt |
| 3241 | gatacgcttt ttagaaatat gcccagaata gaaaagctat gttggggcac atgtcctgca |
| 3301 | aatatggccc tagaaacaag tgatatggaa tttacttggt gaataagtta taaattccca |
| 3361 | cagaagaaaa atgtgaaaga ctgggtgcta gacaagaagg aagcaggtaa agggatagtt |
| 3421 | gctttgtcat ccgtttttaa ttattttaac tgacccttga caatcttgtc agcaatatag |
| 3481 | gactgttgaa caatcccggt gtgtcaggac ccccaaatgt cacttctgca taaagcatgt |
| 3541 | atgtcatcta ttttttcttc aataaagaga tttaatagcc atttcaagaa atcccataaa |
| 3601 | gaacctctct atgtcccttt ttttaattta aaaaaaatga ctcttgtcta atattcgtct |
| 3661 | ataagggatt aattttcaga ccctttaata agtgagtgcc ataagaaagt caatatatat |
| 3721 | tgtttaaaag atatttcagt ctaggaaaga ttttccttct cttggaatgt gaagatctgt |
| 3781 | cgattcatct ccaatcatat gcattgacat acacagcaaa gaagatatag gcagtaatat |
| 3841 | caacactgct atatcatgtg taggacattt cttatccatt ttttctcttt tacttgcata |
| 3901 | gttgctatgt gtttctcatt gtaaaaggct gccgctgggt ggcagaagcc aagagacctt |
| 3961 | attaactagg ctatattttt cttaacttga tctgaaatcc acaattagac cacaatgcac |
| 4021 | ctttggttgt atccataaag gatgctagcc tgccttgtac taatgtttta tatattaaaa |
| 4081 | aaaaaaaatc tatcaaccat ttcatatata tcccactact caaggtatcc atggaacatg |
| 4141 | aaagaataac atttatgcag aggaaaaaca aaaacatccc tgaaaatata cacactcata |
| 4201 | cacacacacg cacaggggaa taaaataaga aaatcatttt cctcaccata gacttgatcc |
| 4261 | catccttaca acccatcctt ctaacttgat gtgtataaaa tatgcaaaca tttcacaaat |
| 4321 | gttctttgtc atttcaaaat actttagtat atcaatatca gtagatacca gtgggtggga |
| 4381 | aagggtcatt acatgaaaat atgaagaaat agccatatta gttttttaac ctgcaatttg |
| 4441 | cctcagcaac aaagaaaaag tgaattttta atgctgaaga taaagtaagc taaagtacca |
| 4501 | gcagaagcct tggctattta tagcagttct gacaatagtt ttataagaac atgaagagaa |
| 4561 | cagaatcact tgaaaatgga tgccagtcat ctcttgttcc cactactgaa ttcttataaa |
| 4621 | gtggtggcaa gatagggaag ggataatctg agaattttta aaagatgatt taatgagaag |
| 4681 | aagcacaatt ttgattttga tgagtcactt tctgtaaaca atcttggtct atctttaccc |
| 4741 | ttatacctta tctgtaattt accatttatt gtatttgcaa agctagtatg gtttttaatc |
| 4801 | acagtaaatc ctttgtattc cagactttag ggcagagccc tgagggagta ttattttaca |
| 4861 | taacccgtcc tagagtaaca ttttaggcaa cattcttcat tgcaagtaaa agatccataa |
| 4921 | gtggcatttt acacggctgc gagtattgtt atatctaatc ctattttaaa agatttttgg |
| 4981 | taatatgaag cttgaatact ggtaacagtg atgcaatata cgcaagctgc acaacctgta |
| 5041 | tattgtatgc attgctgcgt ggaggctgtt tatttcaacc tttttaaaaa ttgtgttttt |
| 5101 | tagtaaaatg gcttattttt tcccaaaggt ggaatttagc attttgtaat gatgaatata |
| 5161 | aaaatacctg tcatccccag atcatttaaa agttaactaa agtgagaatg aaaaaacaaa |
| 5221 | attccaagac actttttaaa agaatgtctg ccctcacaca cttttatgga tttgtttttc |
| 5281 | ttacataccc atcttttaac ttagagatag cattttttgc cctctttatt ttgttgtttg |
| 5341 | tttctccaga gagtaaacgc tttgtagttc tttctttaaa aaacattttt tttaaagaag |
| 5401 | aagaagccac ttgaaccctc aataaaggct gttgcctaag catggcatac ttcatctgtt |
| 5461 | ctcatttgtg ccatctgccg tgatgtcgtc acttttatgg cgttaatttc ctgccactac |
| 5521 | agatcttttg aagattgctg gaatactggt gtctgttaga atgcttcaga ctacagatgt |
| 5581 | aattaaaggc ttttcttaat atgttttaac caaagatgtg gagcaatcca agccacatat |
| 5641 | cttctacatc aaatttttcc attttggtta ttttcataat ctggtattgc attttgcctt |
| 5701 | ccctgttcat acctcaaatt gattcatacc tcagtttaat tcagagaggt cagttaagtg |
| 5761 | acggattctg ttgtggtttg aatgcagtac cagtgttctc ttcgagcaaa gtagacctgg |
| 5821 | gtcactgtag gcataggact tggattgctt cagatggttt gctgtatcat ttttcttctt |
| 5881 | tttcttttcc tggggacttg tttccattaa atgagagtaa ttaaaatcgc ttgtaaatga |
| 5941 | gggcatacaa gcatttgcaa caaatattca aatagaggct cacagcggca taagctggac |
| 6001 | tttgtcgcca ctagatgaca agatgttata actaagttaa accacatctg tgtatctcaa |
| 6061 | gggacttaat tcagctgtct gtagtgaata aaagtgggaa attttcaaaa gtttctcctg |
| 6121 | ctggaaataa ggtataattt gtattttgca gacaattcag taaagttact ggctttctta |
| 6181 | gtgaaaaaaa aaaa |

(SEQ ID NO: 9) GenBank Accession NM_001131005.2 (GI: 301069378), incorporated herein by reference.

Exemplary regions or fragments of MEF2C nucleic acid sequences include bases 331-333 (upstream in-frame stop-codon), 484-687 (exon), and 1364-1499 (exon).

### MESP1 (Mesoderm Posterior Protein 1) Basic Helix Loop Helix

MESP1 is a cardiac specific transcription factor expressed in white blood cells (WBCs).

### Human MESP1 Amino Acid Sequence

| | |
|---|---|
| 1 | maqplcppls eswmlsaawg ptrrpppsdk dcgrslvssp dswgstpads pvasparpgt |
| 61 | lrdprapsvg rrgarssrlg sgqrqsaser eklrmrtlar alhelrrflp psvapagqsl |
| 121 | tkietlrlai ryighlsavl glseeslqrr crqrgdagsp rgcplcpddc paqmqtrtqa |
| 181 | egqgqgrglg lvsavragas wgsppacpga raapeprdpp alfaeaacpe gqamepspps |
| 241 | pllpgdvlal letwmplspl ewlpeepk |

(SEQ ID NO: 10) GenBank Accession NP_061140.1 (GI: 14149724), incorporated herein by reference.

Exemplary regions or fragments of MESP1 include residues 83-140 (helix-loo-helix domain), residues 83-122 (DNA binding region), 97-140 (dimerization interface), region 16.-167, and region 182-185.

Human MESP1 nucleotide sequence. The start and stop codons of the coding sequence are bold and underlined.

| | |
|---|---|
| 1 | tggaaggggc cacttcacac ctcgggctcg gcataaagcg gccgccggcc gccggccccc |
| 61 | agacgcgccg ccgctgccat ggcccagccc ctgtgcccgc cgctctccga gtcctggatg |
| 121 | ctctctgcgg cctggggccc aactcggcgg ccgccgccct ccgacaagga ctgcggccgc |
| 181 | tccctcgtct cgtccccaga ctcatggggc agcaccccag ccgacagccc cgtggcgagc |
| 241 | cccgcgcggc caggcaccct ccgggacccc cgcgccccct ccgtaggtag gcgcggcgcg |
| 301 | cgcagcagcc gcctgggcag cgggcagagg cagagcgcca gtgagcggga gaaactgcgc |
| 361 | atgcgcacgc tggcccgcgc cctgcacgag ctgcgccgct ttctaccgcc gtccgtggcg |
| 421 | cccgcgggcc agagcctgac caagatcgag acgctgcgcc tggctatccg ctatatcggc |
| 481 | cacctgtcgg ccgtgctagg cctcagcgag gagagtctcc agcgccggtg ccggcagcgc |
| 541 | ggtgacgcgg ggtcccctcg gggctgcccg ctgtgccccg acgactgccc cgcgcagatg |
| 601 | cagacacgga cgcaggctga ggggcagggg caggggcgcg ggctgggcct ggtatccgcc |
| 661 | gtccgcgccg gggcgtcctg gggatccccg cctgcctgcc ccggagcccg agctgcaccc |
| 721 | gagccgcgcg acccgcctgc gctgttcgcc gaggcggcgt gccctgaagg gcaggcgatg |
| 781 | gagccaagcc caccgtcccc gctccttccg ggcgacgtgc tggctctgtt ggagacctgg |
| 841 | atgcccctct cgcctctgga gtggctgcct gaggagccca agtgacaagg gacaactgac |
| 901 | gccgtctctg tgagcaccga ggctttttgg cctcagcacc ttcgaagtgg ttccttggca |
| 961 | gactgccttt cctggaagag ggcacgggcg atcccgacgg gggcattcct gcgggtgaga |
| 1021 | gccgtcccca ccgcggcggc ccttctcagc ccctccctcc atggagggac ccatagggct |
| 1081 | agacactttg aggcaagcag gaggctctgc ctaatgtgaa tttatttatt tgtgaataaa |
| 1141 | ctgtactggt gtcagttggc aaaaaaaaaa aaaaaaaaaa aaaa |

(SEQ ID NO: 11) GenBank Accession NM_018670.3 (GI: 219842275), incorporated herein by reference.

Exemplary regions for MESP1 include bases 622-633, and 802-1162.

### Binding Ligands for Biomarkers

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab} and F_{(ab′)2} fragments, and an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM; preferably ≦100 nM and most preferably ≦10 nM.

Antibodies can be produced according to any method known in the art.

Methods of preparing monoclonal antibodies are known in the art. For example, monoclonal antibodies may be prepared using hybridoma methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a full length protein or a fragment thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (see pp. 59-103 in Goding (1986) Monoclonal Antibodies: Principles and Practice Academic Press) Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some examples the antibodies to an epitope for an interested protein as described herein or a fragment thereof are humanized antibodies. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al. 1986. Nature 321:522-525; Riechmann et al. 1988. Nature 332:323-329; Presta. 1992. Curr. Op. Struct. Biol. 2:593-596). Humanization can be essentially performed following methods of Winter and co-workers (see, e.g., Jones et al. 1986. Nature 321:522-525; Riechmann et al. 1988. Nature 332:323-327; and Verhoeyen et al. 1988. Science 239:1534-1536), by substituting rodent CDRs or CDR sequences for the corresponding sequences of a human antibody. Accordingly, such humanized antibodies are chimeric antibodies (e.g., U.S. Pat. No. 4,816,567), wherein substantially less than an intact human variable domain has been substituted by the corresponding sequence from a non-human species.

In another examples the antibodies to an epitope of an interested protein as described herein or a fragment thereof are human antibodies. Human antibodies can also be produced using various techniques known in the art, including phage display libraries (Hoogenboom and Winter. 1991. J. Mol. Biol. 227:381-388; Marks et al. 1991. J. Mol. Biol. 222:581-597) or the preparation of human monoclonal antibodies (e.g., Cole et al. 1985. Monoclonal Antibodies and Cancer Therapy Liss; Boerner et al. 1991. J. Immunol. 147(1):86-95). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in most respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al. 1992. Bio/Technology 10:779-783; Lonberg et al. 1994. Nature 368:856-859; Morrison. 1994. Nature 368:812-13; Fishwild et al. 1996. Nature Biotechnology 14:845-51; Neuberger. 1996. Nature Biotechnology 14:826; Lonberg and Huszar. 1995. Intern. Rev. Immunol. 13:65-93. U.S. Pat. No. 6,719,971 also provides guidance to methods of generating humanized antibodies.

Exemplary antibodies against HuR (ELAVL1) protein include, but are not limited to, antibodies obtained from “antibodies online” (e.g., Cat. No. ABIN577055; Cat. No. ABIN659117; Cat. No. ABIN652911; Cat. No. ABIN396456; Cat. No. ABIN307186, and more can be found at its website www.antibodies-online.com), antibodies obtained from “abcam.com” (e.g., ab54987, ab135740, ab137471, ab136542, and more can be found at its website www.abcam.com), antibodies obtained from Santa Cruz Biotech (e.g., sc-5261, sc-20694, sc-374285 and more can be found at its website www.scbt.com); any commercially available antibodies against HuR, and any antibodies that are generated by known method in the art utilizing the full-length protein or a fragment of human HuR (e.g., residues 243-326, residues 180-183, residues 157-167, residues 146-156, residues 141-143, residues 107-185, residues 106-189, residues 19-96, residues 19-326, any fragment or full length of SEQ ID NO 1).

Exemplary antibodies against HuB protein include, but are not limited to, antibodies obtained from Sigma-Aldrich (e.g., H1538), antibodies obtained from LifeSpan BioSciences (e.g., LS-B9943, and more can be found at its website www.lsbio.com), antibodies obtained from Santa Cruz Biotech (e.g., sc-5982), any commercially available antibodies against HuB, and any antibodies that are generated by known method in the art utilizing the full length or a a fragment of human HuB (e.g., residues 38-115, residues 40-115, residues 120-209, residues 126-203, residues 239-251, residues 276-354, any fragment or full length of SEQ ID NO 3).

Exemplary antibodies against human SCN5A protein include, but are not limited to, antibodies obtained from “Thermo Scientific online” (e.g., Cat. No. PAS-34190; Cat. No. MA1-27429; Cat. No. PAS-39462; Cat. No. PAS-36074, and more can be found at its website www.pierce-antibodies.com), antibodies obtained from “abcam.com” (e.g., ab53724, ab62388, ab116706, ab86321, and more can be found at its website www.abcam.com), antibodies obtained from Santa Cruz Biotech (e.g., sc-271255, sc-81631, sc22758, sc23174, and more can be found at its website www.scbt.com); any commercially available antibodies against SCN5A, and any antibodies that are generated by known method in the art utilizing the full length or a fragment of human SCN5A (e.g., residues 159-412, residues 159-178, residues 842-862, residues 1201-1224, any fragment or full length of SEQ ID NO 12).

Exemplary antibodies against human MESP1 protein include, but are not limited to, antibodies obtained from “Thermo Scientific online” (e.g., Cat. No. MA5-15645; and more can be found at its website www.pierce-antibodies.com), antibodies obtained from “abcam.com” (e.g., ab77013, ab171427, ab129387, ab86419, and more can be found at its website www.abcam.com), antibodies obtained from Santa Cruz Biotech (e.g., sc-163077, sc-163078, sc-163074, sc-163076, and more can be found at its website www.scbt.com); any commercially available antibodies against MESP1, and any antibodies that are generated by known method in the art utilizing the full length or a fragment of human MESP1 (e.g., residues 83-140, residues 83-122, residues 97-140, residues 16.-167, residues 182-185, any fragment or full length of SEQ ID NO 10).

Exemplary antibodies against human MEF2C protein include, but are not limited to, antibodies obtained from “Thermo Scientific online” (e.g., Cat. No. MA5-17119; Cat. No. PAS-28247; Cat. No. PAS-34581; Cat. No. PAS-13287, and more can be found at its website www.pierce-antibodies.com), antibodies obtained from “abcam.com” (e.g., ab64644, ab197070, ab191428, and more can be found at its website www.abcam.com), antibodies obtained from Santa Cruz Biotech (e.g., sc-365862, sc-13266, sc-13268, and, more can be found at its website www.scbt.com); any commercially available antibodies against MEF2C, and any antibodies that are generated by known method in the art utilizing the full length or a fragment of human MEF2C (e.g., residues 2-38, residues 3-57, residues 4-31, residues 21-73, residues 87-134, residues 107-134, residues 110-156, residues 271-278, residues 368-399, any fragment or full length of SEQ ID NO 8).

### Example 1: Human Heart Failure is Associated with Sodium Channel mRNA Degradation Mediated by Reduced Levels of HU Proteins

Blood samples of control and heart failure patients with evidence of appropriate event-driven therapy [ICD(+)Event] were collected in PAXgene® Blood RNA tubes (BD Biosciences) following the manufacturer's procedure. Total RNA was isolated using the PAXgene Blood RNA isolation kit and then converted to cDNA using the High Capacity cDNA Reverse Transcription Kit (Qiagen, Valencia, Calif.). Real-time quantitative PCR (q-PCR) analysis was performed using 7500-Fast Real-Time PCR Systems with specific primers and Fast SYBR® Green Master Mix (Applied Biosystems).

Human fetal cardiomyocyte cell line RL14 was purchased from ATCC and was maintained in DMEM/F-12 supplemented with 12.5% fetal bovine serum. Cells were transfected with SuperFect® Transfection Reagent (Qiagen) for exogenous over expression experiments, and were transfected with Lipofectamine® RNAiMAX™ (Life Technologies) for siRNA knockdown experiments. Human HuR tagged with HA was cloned into pcDNA 3.1 using standard cloning procedures and confirmed by sequencing. Scrambled siRNA (control) and siRNA against Human HuR were purchased from Life Technologies. Total RNA was isolated by using RNeasy® Plus Mini kit (Qiagen) and was reverse transcribed with SuperScript® III Reverse Transcriptase and a random primer (Life Technologies) followed by RT-qPCR. For transcription inhibition, actimomycin D was added at 5 mg/mL, and the remaining levels of indicated mRNAs were measured by RT-qPCR analysis.

A ribonucleoprotein (RNP) complex immunoprecipitation (RIP) assay was performed using formaldehyde as a reversible cross-linker to study RNA-protein interactions. Other stabilizing agents such as reversible or non-reversible crosslinking agents were used to yield a durable protein-mRNA complex. Endogenous HuR-mRNA complexes were precipitated with an anti-HuR antibody (Santa Cruz Biotechnology). HuR-bound RNA was reverse transcribed (RT) using SuperScript reverse transcriptase (Life Technologies), and the specific mRNAs were detected by RT-PCR. Western blot analysis was performed following a standard procedure. Human heart total RNA was purchased from Zyagen Laboratories.

HuR, a major member of HU protein family, was found to be expressed in human heart and in circulating WBC such as monocytes and T cells. HuR mRNA was decreased in peripheral white blood cells of heart failure patients with implanted cardioverter-defibrillator (ICD) and evidence of appropriate event-driven therapy.

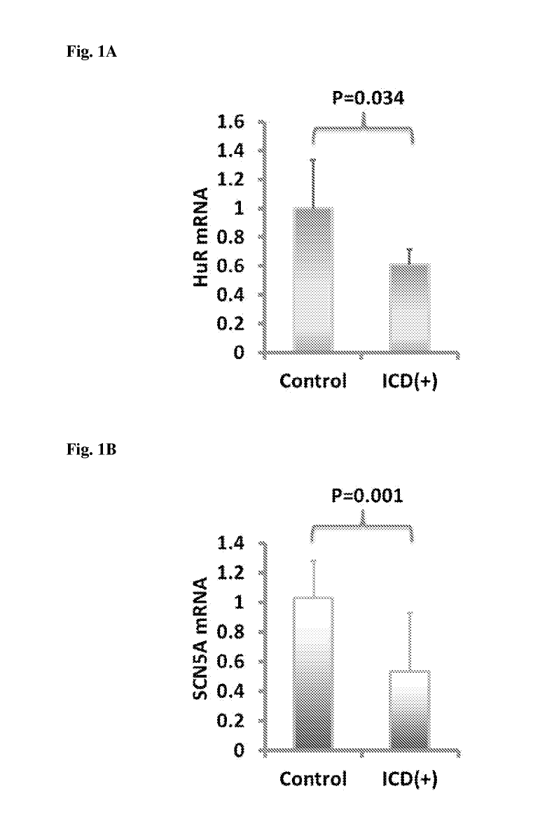

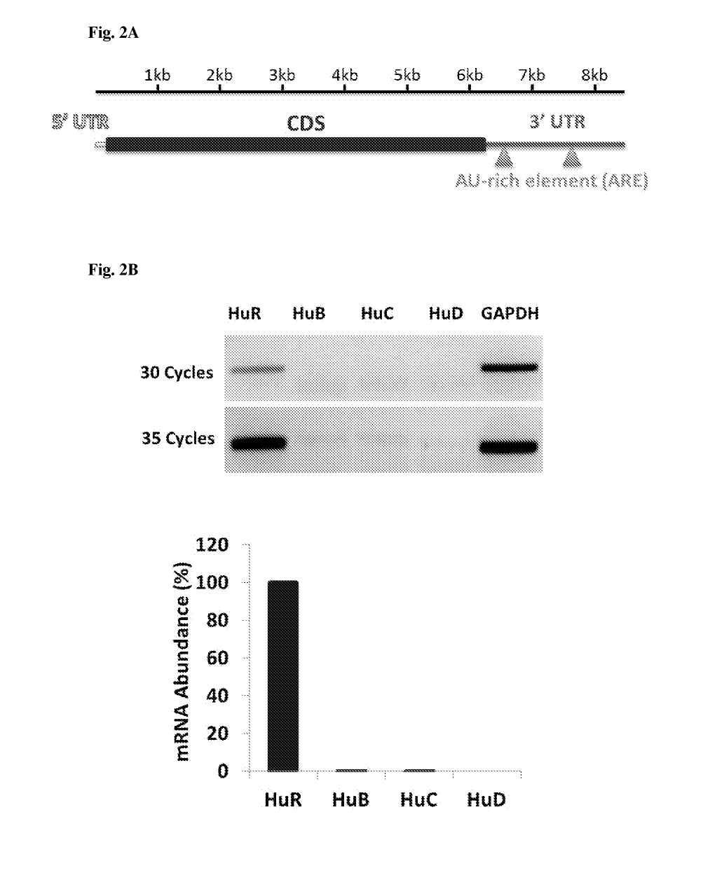

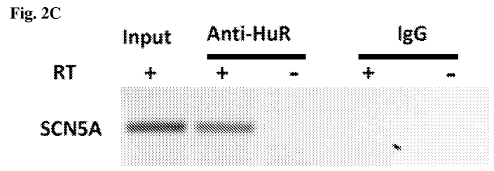

The mRNA of the sodium channel gene, SCN5A, also was decreased in these patients. SCN5A expression was found to be correlated with HuR expression in human cardiomyocytes. HuR bound to and stabilized SCN5A mRNA. (FIGS. 1A-B, 2A-C, 3A-C, and 4A-B). These data indicate that RNA binding proteins such as HuR regulate SCN5A gene expression by stabilizing its mRNA and that reduced HuR (compared to normal control level) is a circulating marker for reduced SCN5A and increased arrhythmic risk.

### Example 2: Use of HU Binding Reagents to Differentiate Between High Risk Patients and Low Risk Patients

A method of identifying a subject with who is at risk of arrhythmia or SCD by detecting and quantifying an HU protein using an HU protein binding agent such as an isolated/purified monoclonal antibody, e.g., a non-naturally occurring antibody that is specific for a human protein but raised in a non-human animal such as mouse, rat, rabbit or goat anti-HuR. A complex between an isolated HU-specific monoclonal antibody and a patient cell-derived HU protein is detected using known methods, e.g., ELISA. The level of the HU protein in the test sample is compared to a reference level (representative of a normal level or range) of the protein, and a lower level is indicative of a clinically relevant risk of heart failure that merits medical intervention.

Alternatively, the method is carried out by detecting HU nucleic acid levels, e.g., by processing patient cell-derived RNA to yield cDNA and determining the level/amount of cDNA in patient-derived WBCs such as monocytes, macrophages, or T cells. Complementary DNA (cDNA) is non-naturally occurring DNA that is synthesized from a messenger RNA (mRNA) template in a reaction catalyzed by the enzymes reverse transcriptase and DNA polymerase using methods well known in the art.

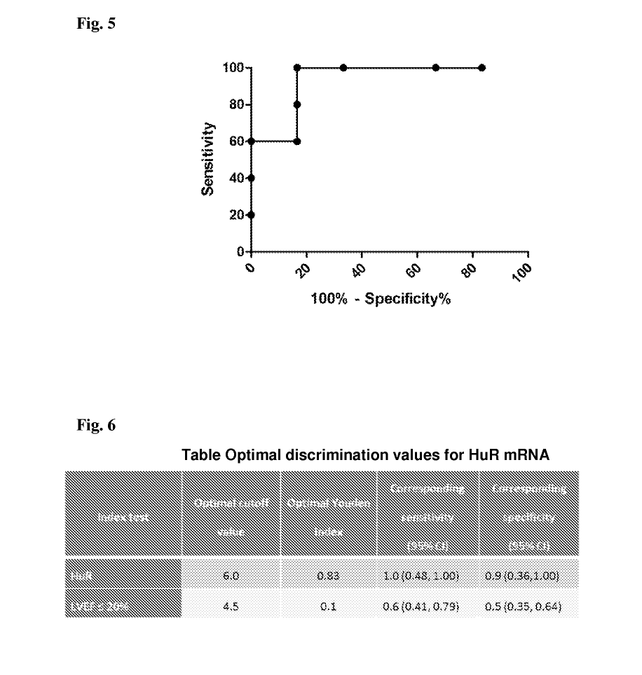

Based on ROC analysis, a cutoff level and reference level were defined (FIGS. 5 and 6).

### Example 3: Patients with BrS Show Reduced SCN5A, HuR and MEF2C in WBC

Blood samples were collected in BD vacutainer 9NC 0.129M (BD Biosciences). White blood cells were separated using Lympholyte-H sterile liquid. Total RNA was isolated by using Trizol® (Life Technologies) and Direct-Zol™ RNA MiniPrep Kit and was reverse transcribed with QuatntiTect Reverse Transcription Kit (Qiagen). Twenty five BrS patients (20 with SCN5A mutation and 5 without SCN5A mutation) were included in this study.

The human fetal cardiomyocyte cell line RL14 was purchased from ATCC. Cells were transfected with SuperFect® Transfection Reagent (Qiagen) according to the manufacturer's instruction. Total RNA was isolated by using RNeasy® Plus Mini kit (Qiagen) and was reverse transcribed with SuperScript® III Reverse Transcriptase and random primer (Life Technologies).

Inducible human HuR and MEF2C constructs were cloned using standard cloning procedures and confirmed by sequencing. Real-time quantitative PCR (Q-PCR) analysis was performed using 7500-Fast Real-Time PCR Systems with specific primers, Probes and TaqMan® Gene Expression Master Mix (Applied Biosystems).

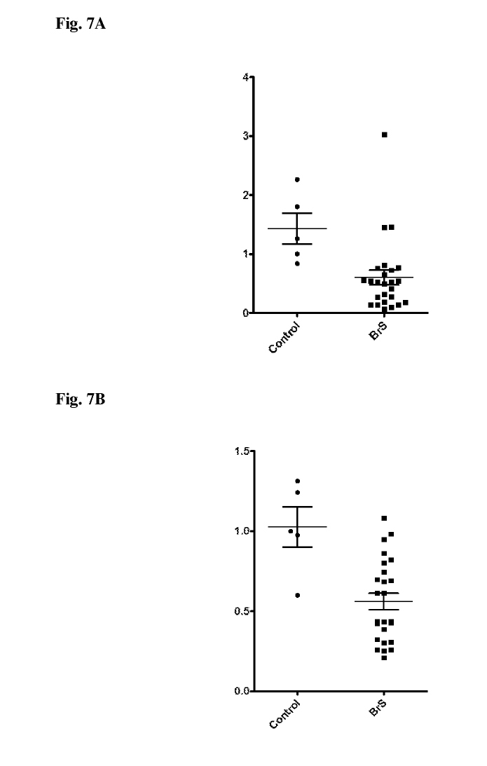

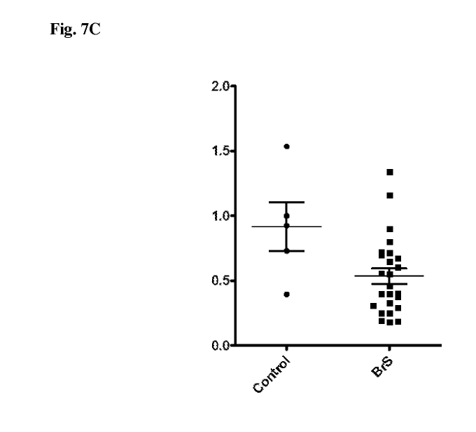

WBC levels of SCN5A decreased (58.04%, P=0.01) in BrS patients (0.62±0.62) compared to the normal control group (1.43±0.59) (FIG. 7A). The SCN5A mRNA stabilizing protein, HuR, was significantly lower (45.63%, P=0.001) in BrS patients (0.56±0.26) compared to the control group (1.03±0.28) (FIG. 7B).

MEF2C, a cardiac transcription factor, was also decreased (41.3%, P=0.02) in BrS (0.54±0.30) compared to the control group 0.92±0.42) (FIG. 7C). Expression of SCN5A mRNA correlated with the expression of HuR and MEF2C mRNA in white blood cells. Overexpression of MEF2C in human fetal cardiomyocytes increased SCN5A mRNA expression, and knockdown of MEF2C reduced the SCN5A mRNA level. Overexpression of HuR in human fetal cardiomyocytes increased, while knockdown of HuR reduced, SCN5A mRNA expression.

### Example 4: HuR Regulates SCN5A Expression in Cardiomyocytes

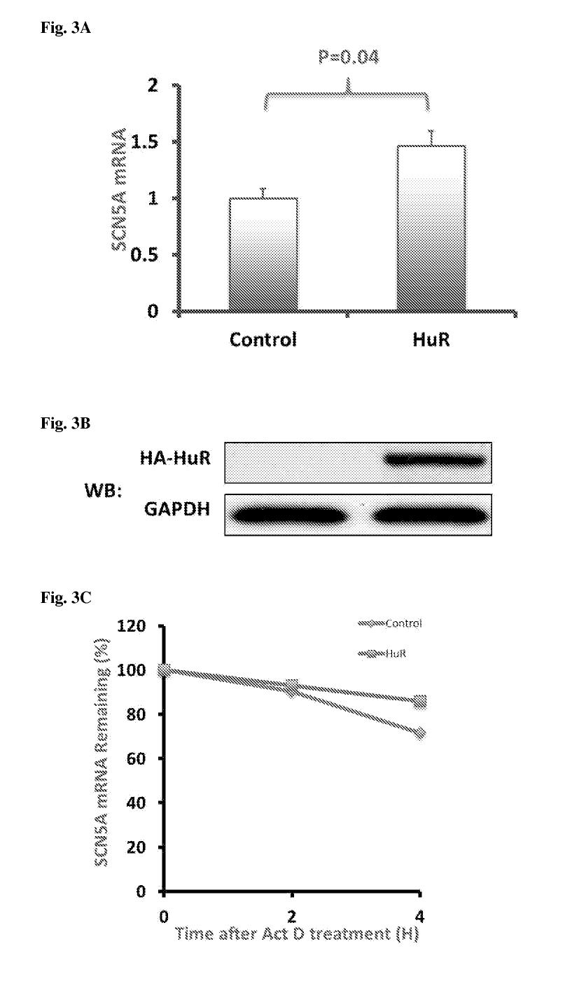

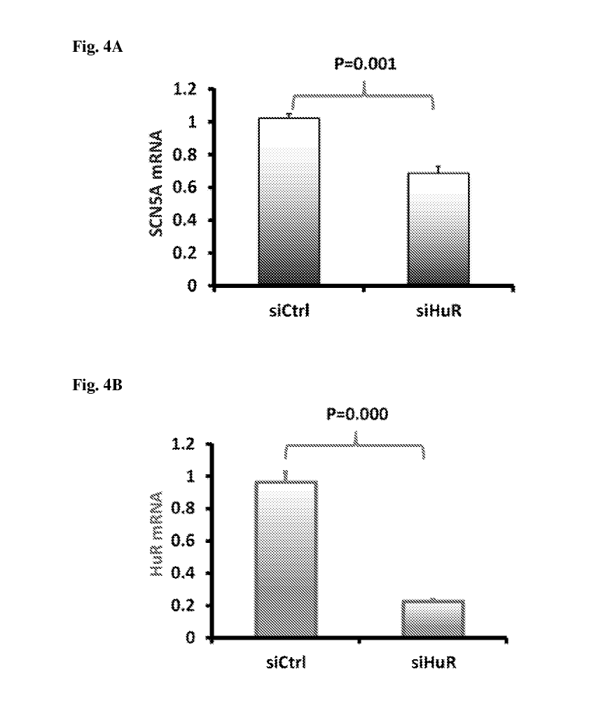

Cells were transfected with either an overexpression construct of HuR or siRNA duplexes of HUR (siHuR) (FIGS. 3A and 4A). Cells were harvested and extracted for total cellular RNA 48 hours after transfection and changes in SCN5A and HuR mRNA levels were determined by RT-qPCR using β-actin mRNA for normalization. Overexpression of HuR increased SCN5A mRNA by 46.3% compared with control group (P=0.04) (FIG. 3A). SCN5A mRNA decreased 32.5% (P=0.001) in cells transfected with siRNA of HuR (siHuR) compared with those transfected with scramble siRNA (FIG. 4A).

### Example 5: The Expression of SCN5A Correlates with the Expression of HuR and MEF2C in WBC

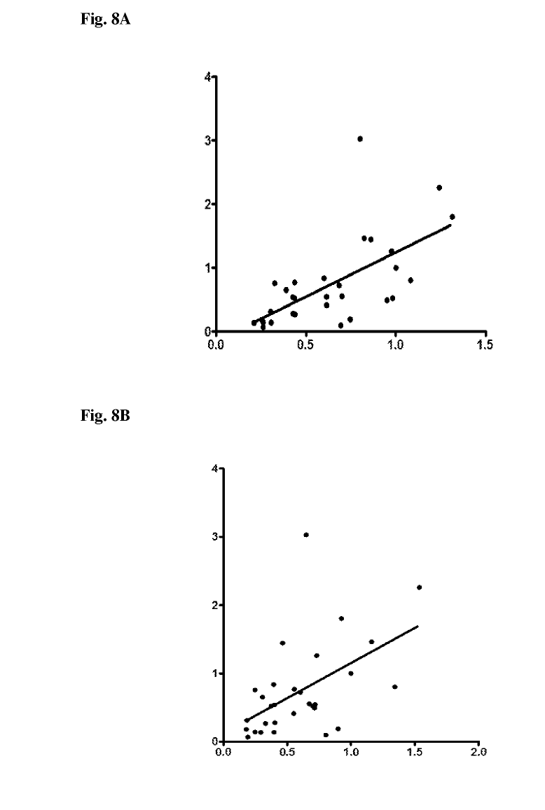

HuR mRNA expression correlated with the expression of SCN5A in white blood cells (R²=0.402, P=0.000) (FIG. 8A). MEF2C mRNA expression correlated with the expression of SCN5A in white blood cells (R²=0.268, P=0.003) (FIG. 8B).

### Example 6: MEF2C Regulates SCN5A Expression in Cardiomyocytes

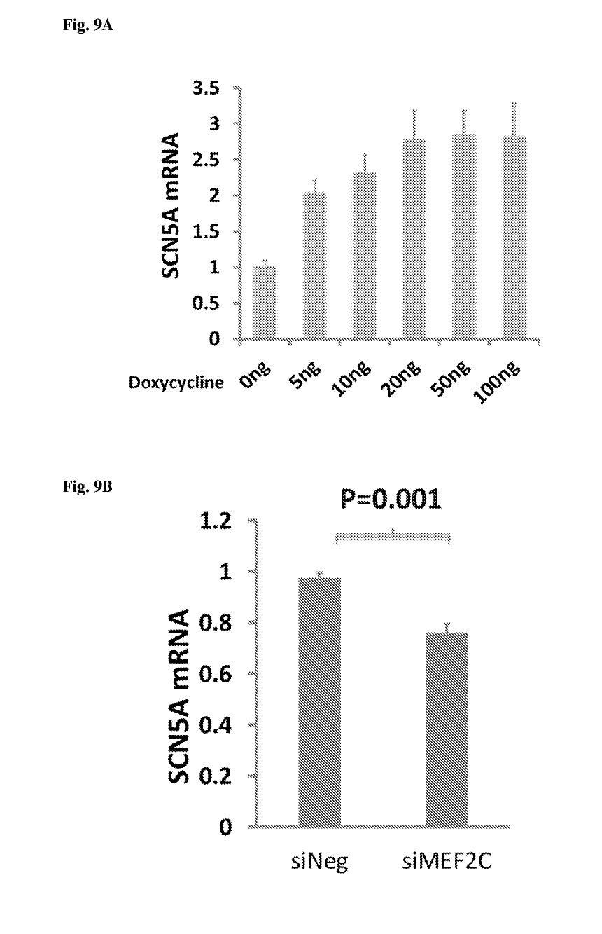

Cells were transfected with either inducible overexpression construct of MEF2C or siRNA duplexes of MEF2C (siMEF2C). MEF2C increased SCN5A mRNA expression in a doxcycline dose-dependent manner (FIG. 9A). SCN5A mRNA decreased 22.3% (P=0.001) in cells transfected with siRNA of MEF2C (siMEF2C) compared with those transfected with scramble siRNA (siCtrl) (FIG. 9B).

### Example 7: mRNA Expression of MESP1 in WBCs was Decreased in Both SCN5A(−) Patients and SCN5A(+) Patients

The mRNA expression of MES1P1 in WBCs was decreased in both SCN5A(−) (P=0.012 vs. control) and SCN5A(+) (P=0.000 vs. control) groups. No difference was observed between the two BrS groups in MESP1 expression (P=0.215). The area under the ROC analysis curve for prediction of BrS using MESP1 levels was 0.775 (95% CI: 0.668-0.882, asymptotic Sig.=0.000). At the optimal cutoff, the corresponding maximum sensitivity and specificity were 0.62 (95% CI: 0.47-0.76) and 0.88 (0.69-0.97) respectively (and summarized in the tables below).

Area under the Receiver Operating Characteristics (ROC) prediction of BrS using MESP1 levels:

| Index Test | mRNA levels in WBCs (95% CI) | Asymptotic Sig. |
|---|---|---|
| MESP1 | 0.775 (0.668-0.882) | 0.000 |

The diagnostic odds ratio (DOR) of MESP1 for BrS diagnosis was 11.96 (95% CI: 5.79-24.73). The assessment of the mRNA levels in blood SCN5A, MEF2C and HuR were useful for predicting BrS patients with an SCN5A mutation. The area under the ROC analysis curve for prediction of BrS with an SCN5A mutation using SCN5A, MEF2C and HuR mRNA levels in WBCs was 0.847 (95% CI 0.752-0.942, asymptotic Sig.=0.000), 0.685 (95% CI 0.542-0.828, asymptotic Sig.=0.016) and 0.777 (95% CI 0.652-0.902, asymptotic and HuR for SCN5A(+) BrS diagnosis was 17.5 (95% CI: 8.06-37.86), 4.9 (95% CI: 2.61-9.17) and 23.5 (95% CI: 9.39-8.80), respectively (and summarized in the tables below).
Area under the DOR (diagnostics odds ratio) analysis for BrS diagnosis (at the optimal cut off, with SCN5A mutation):

| Index Test | mRNA levels in WBCs (95% CI) |
|---|---|
| SCN5A mutation | 17.5 (8.06-37.86) |
| MEF2C | 4.9 (2.61-9.17) |
| HuR | 23.5 (9.39-58.80) |
| MESP1* | 11.96 (5.79-24.73) |

*Circulating MESP1 may be used as a biomarker

Area under the ROC analysis (prediction of BrS with an SCN5A mutation) in WBCs:

| Index Test | mRNA levels in WBCs (95% CI) | Asymptotic Sig. |
|---|---|---|
| SCN5A mutation | 0.847 (0.752-0.942) | 0.000 |
| MEF2C | 0.685 (0.542-0.828 | 0.016 |
| HuR | 0.777 (0.652-0.902) | 0.000 |

Area under the Receiver Operating Characteristics (ROC) prediction of BrS using MESP1 levels:

| | Corresponding | Corresponding |
|---|---|---|
| Index Test | Sensitivity (95% CI) | Specificity (95% CI) |
| MESP1 | 0.62 (0.47-0.76) | 0.88 (0.69-0.97) |

The results indicated that assessment of circulating MESP1 is useful as a biomarker for BrS diagnosis, while decreased SCN5A, MEF2C and HuR mRNA in WBCs is associated with BrS patients with an SCN5A mutation. The results also indicated that decreased expression of SCN5A, MEF2C, MESP1, and HuR may be pathophysiologically related to BrS.

MESP1 is used as a biomarker for BrS diagnosis; and may be pathophysiologically related to BrS.

### Example 7: In Vitro Diagnostic (Clinical) Screening Assays with Targeted Population

In vitro diagnostics tests are used that detect diseases, conditions, or infections. Some tests are used in laboratory or other health professional settings and other tests are for consumers to use at home. The Youden's index is used to assess the performance of a diagnostic test (alternatively, the Youden's index is a way of summarizing the performance of a diagnostic test). The values range from zero to one, wherein a zero value indicates that the test gives the same proportion of positive results for groups with and without the disease, and a value of 1 indicate that there are no false positives or false negatives. The optimal Youden index for HuR mRNA is 0.83 (with a corresponding sensitivity of 1.0, 95% CI and a corresponding specificity of 0.9, 95% CI) (FIG. 6).

### OTHER EMBODIMENTS

While the invention has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank, NCBI, UniProt, or other submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the disclosure encompassed by the appended claims.

What is claimed:

1. A method for evaluating arrhythmic risk or assessing the severity of heart failure in a subject in need thereof, comprising:
providing a test sample from said subject, wherein said sample comprises a circulating cell or a bodily fluid,
performing a reaction in vitro to yield a complex comprising an HU protein or an HU nucleic acid and binding agent, and
detecting the complex, wherein a decrease in the level of said complex compared to a normal control indicates an increased risk of arrhythmia or an increased severity of heart failure.

2. The method of claim 1, further comprising computing a level of said HU protein or HU nucleic acid,
wherein at least 30% decrease in the level of said protein or nucleic acid compared to a normal control indicates a high risk of arrhythmia or an increased severity of heart failure.

3. The method of claim 1, further comprising computing a level of an sodium channel, voltage gated, type V alpha protein (SCN5A), a mesoderm posterior protein 1 protein (MESP1), or a myocyte enhancer factor-2 protein (MEF2C) with a binding agent, wherein a decrease in the level of said protein compared to a normal control indicates a high risk of arrhythmia or an increased severity of heart failure.

4. The method of claim 1, further comprising computing a level of an SCN5A variant, wherein an increase in the level of the variant compared to a normal control indicates a high risk of arrhythmia or an increased severity of heart failure.

5. The method of claim 1, wherein the binding agent comprises an antibody or a fragment thereof, a detectable protein, or a fragment thereof, or a nucleic acid molecule.

6. (canceled)

7. (canceled)

8. The method of claim 1, wherein said HU protein or HU nucleic acid comprises an amino acid sequence or nucleic acid sequence of human HuR (SEQ ID NO: 1 and SEQ ID NO: 2, respectively) or HuB (SEQ ID NO: 3 and SEQ ID NO: 4, respectively), or fragment thereof.

9.-15. (canceled)

16. The method of claim 4, wherein said SCN5A variant comprises an amino acid sequence or nucleic acid sequence of human SCN5A, comprising SEQ ID NO: 5, SEQ ID NO: 6, SEQ ID NO: 7.

17.-22. (canceled)

23. The method of claim 3, wherein said MESP1 protein or MESP1 nucleic acid comprises an amino acid sequence or nucleic acid sequence of human MESP1 (SEQ ID NO: 10 and SEQ ID NO: 11, respectively), or fragment thereof.

24.-29. (canceled)

30. The method of claim 3, wherein said MEF2C protein or MEF2C nucleic acid comprises an amino acid sequence or nucleic acid sequence of human MEF2C (SEQ ID NO: 8 and SEQ ID NO: 9, respectively), or fragment thereof.

31.-37. (canceled)

38. The method of claim 1, further comprising contacting said protein with a stabilizing compound to yield a durable protein/mRNA complex.

39. The method of claim 1, wherein said test sample comprises plasma, whole blood, serum, saliva or urine.

40. (canceled)

41. (canceled)

42. The method of claim 1, further comprising repeating said providing, performing, and detecting steps over time, wherein a progressive decrease over time in the level of said protein and/or said nucleic acid indicates an increase in the severity of cardiac impairment.

43. The method of claim 1, wherein said test sample comprises a population of enriched white blood cells.

44. The method of claim 43, wherein said population comprises a buffy coat fraction of total white blood cells.

45.-48. (canceled)

49. The method of claim 1, further comprising identifying said subject with a high risk of arrhythmia or an increased severity of heart failure, and administering to said subject a compound for increasing the level of said protein.

50. A kit comprising an agent for detecting level of an HU protein for detecting arrhythmic risk,
wherein the agent binds to an HU protein or nucleic acid encoding said protein, and yields a complex comprising said HU protein or nucleic acid and said binding agent
and instructions for using the agent for evaluating arrhythmic risk or assessing the severity of heart failure.

51. (canceled)

52. (canceled)

53. (canceled)

54. A method of reducing risk of arrhythmia or severity of heart failure in a subject comprising,
measuring the level of an HU protein in a subject, wherein the decreased level of an HU protein indicates a high risk of arrhythmia or an increased severity of heart failure, and
administering to said subject an active agent for reducing risk of arrhythmia or severity of hearting failure.

55. (canceled)

56. (canceled)

57. A diagnostic test system comprising,
obtaining test results data representing levels of a marker in at least one biological sample,
collecting test results data,
a means for computing an index value from said marker, wherein the index value comprises an arrhythmic risk score or a heart failure risk score, and
a means of reporting the index value.

58. (canceled)

59. (canceled)

60. (canceled)

61. A diagnostic device comprising a solid support and an HU binding agent immobilized on said support.

62. A diagnostic device comprising a plurality of immobilized binding agents, wherein a first binding agent binds to HuR or HuB, a second binding agent binds to SCN5A, a third binding agent binds to MESP1, or a fourth binding agent binds to MEF2C.

63.-65. (canceled)


SEQUENCE LISTING (13 sequences)

SEQ ID NO: 1 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 2 - DNA, 6075 bp, Homo sapiens
ggtcgtgcgcgctgaggaggagccgctgccgccgtcgccgtcgccgccaccgccgccaccgctaccgaggccgagcggagccgttagcgccgcgccgccgccgcctcccgcccgccccggagcagccccgggcccgcccgcccgcatccagatttttgaaaaatacaatgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctatggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcacagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataactcgctcatgcttttttttgtacggaatagataattaagagtgaaggagttgaaacttttcttgttagtgtacaactcattttgcgccaattttcacaagtgtttgtctttgtctgaatgagaagtgagaaggtttttatactctgggatgcaaccgacatgttcaaatgtttgaaatcccacaatgttagaccaatcttaagtttcgtaagttatttcctttaagatatatattaaacagaaatctaagtagaactgcattgactaaccagtccctctggatggtggtgaacctgaagcatgctttaacctctaagactgtctaacacgcgtttcattcaatgtctccacagactgggtagcaaaaaaatcaccttttagttttagtttttaatctaaagatgttagacagatgctgagtgtgcgttttctcaaccgcttcaacattgtaagcgatgtatgctttggttgacaggaagttccttttccaggcaggtcccgttgccacctcctgctcactcagtcccgggctctgccgagtggtcctgggaatggcggcgggcccgtccagcgtgggccaccactggggccgggggccacgggctgcatgctgggcgggccctccagagaaggacacaaacgtgtttcgtaagcccaggcaccaatgggaatggaccaaagagtttcagggaaactccagtatattccagagtcagatctaagctccaggcacgcctgaagatgtgttgctactctgacatcccgagtttctgtccacacattgcatgcacagcgccccacacattggatactgttgttcacgataatttctcccgttttccagagcatttaacatagcttggaggcgtaaaatggctctgtattttaataacacagaaacatttgagcattgtatttctcgcatcccttctcgtgagcgcttagacctttttctattttagtcggattttgttttggaattttgcttttgtatgaacactcagcagaaaagtacttacttcttgccagttatctattaaccaaaacctttgatttgtagttttaaagattaaccctcaaagttctcttcataactgccttgacattttgggttgttctgttctgttaattttcttttgcttttttgtgttttttgtttgtttttacttttgcatttaagaccattaaatttgattttgttttgctcgaattttgttttgttttttattttaccttttctttctttttggctagggaaggtgcaggtggcccagcattcagggaggagtcgtaagatcttaagaaaccaatccttgcctcaagcaaaagcatttctgaatctgtgacgcaagaatgtgcagttacaggctggtggcttttaaaccaggagcccggaggaagggtgaaagagaaagcctgtgaaataggcagggccagatcacccaaaactcctcaggactgggatctggcgtttataaataactagtttacagagagaatcacaaacaggataacttagtaccagcagtttttaaccttgacgtgagactaaaacgtgaccgtaggctgttttttagttattgctctcatgagatgatggctgtatttatctgtttatttatacctatttatgtatttatttattgaagtgtgaaattgagcaataggcaggcaccaccgtccccagagcaggtcagcgtctcgagaggcccctggacaattgaggatgcccatcccctcccctttccctgatcttttactgaggggctgtgtgcgcgatccttgcaaattgatgatgttgccatccgtacccaggctgtgtctcataaaagtcggcctggtgccagagaggacctcctttctcccacagaatcccagatcctcaggaaaagccaaaaccgaggcccattgcccggattcgacacaaaagagggtccctgctctgttgcccgagagcagtctgcatcctgggaccagaatgctttcctggaaaaagaagcctttcaggtttccctgggccagcatcttctgatggaaggtgggagccaacacccttctgatggaaggtgggagccaacacccttctgatggaaggtgggagccaacacccttctgatggaaggtgggagccaacacccttctgatggaaggtgggagccaacacccttctgatggaaggcgggattcccgcttctgaaactccccctggagtctcactcccacacatgcccatagctagcattcaacagagaactctgtcttaagcttcaactgtgaaaatgatgacgggcttgtagcacctcagcttctttcctcgcccccttttatctgaatcctatcaattattctgatgctgggacaggtgagaagaaactgtgaagtatatgagcctttgaaagttccctgaagtttctcagttcaggaacattctcattgtatgtggtctccgctgtttgaacagccttctagctaaaaaattccaaagcctttatttgggagtcttagcttgcaagcttgtggaaggatttagcttaacaactgtcactcctgaaaagcaatctctgttccatcaaggttctagttgctggccctgtgtcctcaaagttcattacatcttatcaaggcctgtttgcaaaggggagatcccttttcttaaaaaaggctcaacccaaaagaaaccatttcttaaaaaattttacatagatcagttgtatttctatttagcaaaaatgagtgctctgcttttatttgggaatttcgatgaaaaagcgttcagagtagataatgttcatttatcaaaaatctggtttgggaaataccaaagaggctttgattgaattcccttttgacccgtgtgtaacttcctctggtagttagaccccaggcagctccgaatttgtgaacctgcttcctgatgaattctcccttgttcccccttggctctgccattatttcgttttcagtgtaatttgccaagccgcagttttctgtgctggctgtgcctctagtcgcagctctgtgactgattccctcccgggtgctgagtcccctccccggccaccatcctgcgtgaatatcctgaaattcatgggcttcctcgggggcccgcccgcaggtggtgctgggtgggttccgccaccttctcctggaaggtgagccttttcctggccaagggcagctgccttaacctctgagagtctgcgcttggccttagtcctggagacccagcctccagggactgaaccgtgctgctgttgggagccaagaccggccctttggagccggcagcccaggggtccctgctggatcagagaaatagaagcacccgaagacggttagtggcaattccttgacccggtttgcttccaaatgaaggccatttgtccaccaggcattgaaaagacatgacttacccagtccggcatcggacttgaaaaatcgaaattgacatcactcagctgttacatttcacatccgattcagcccccttttatttccatgtgcttttcgcagccttcctgtgttggatgaaagagaataagaattcagctgacaggaggcctctatcctgtccctccaccccaccctccacctcaatcccctcccatcttccccagacctacctcacctactaggacctgaggcagctccttagcagagacccctggggtttagctgactctgggggtcaggggttcctgtccccaaacttcgcaagacagccctgaagtcacaagtgctttcttttaagtggcattggcaattggcgtgtaatgatggcagtagaatctgaatctcggatcccaggcagggttcacatttccaaacctttttgatttcccctgacctctaatggctggatcctatttttctacaacctttcagtgacatcgttcaggtttctttcttggccatttaaaaaaacaaattttttttttctcacttgtaagtcaccgccagtacctaagttaggctaacggagactttgacaggactggatttttcttccaccagaagagaagccttttccgttggtttggggccacctctttgaccatgaccatgtgatgttccgtttacagtgacttgctttgggggaggggaggctctcttaaccgattcccatgttgtacagtagatggttagaccttttgtatattagtgtgttttaagttattgatttgttttatataaaataatttatttttcaggtgccatttttcattttaactttgtttttacatgggtttgttttcaataaagtctgacactggtgtccaaaagtcaacaataaaatgaatcccattgtgttcttttgaagatgcctatgtaacttttaagctttttaaattattttcagaaaaaaaaaaagaaaagcccttatcagttttccatcagcccattgcctttttattttttttttttaatccttgtgaataaatgttctttagtgttttaggaggaaaaagcaaacctagattttgataacccagaagacttcagattaataaagaagctttgaaagaagaccatttttcaaaattttagtgaagtgtgaatattttttgtcaatggctttctcaaagagaatgaaacttttgcaccattttcagagtttttatagagatgccaaattgatatatttacatgtaatggaaacatgaaaaagttttattaaacaattgttcatagctgtgtagacattttaattcagtttccaaagctctcaaaaaatcgtatttttgaagtacggagtgatgcggtttggggcgtggcttacagttccaacgactcaattgtcccgatactcagttctttctacaggtatcaggttcgtgttaaacgctgtatgttaactatgactggaattctgtgatattttggtaataaatgaagtgggatcattgcgaaaaaaaaaaaaaaaaaa

SEQ ID NO: 3 - protein, 359 aa, Homo sapiens
METQLSNGPTCNNTANGPTTINNNCSSPVDSGNTEDSKTNLIVNYLPQNMTQEELKSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGISRGVGFIRFDKRIEAEEAIKGLNGQKPPGATEPITVKFANNPSQKTNQAILSQLYQSPNRRYPGPLAQQAQRFRLDNLLNMAYGVKRFSPMTIDGMTSLAGINIPGHPGTGWCIFVYNLAPDADESILWQMFGPFGAVTNVKVIRDFNTNKCKGFGFVTMTNYDEAAMAIASLNGYRLGDRVLQVSFKTNKTHKA

SEQ ID NO: 4 - DNA, 3814 bp, Homo sapiens
caataggagggtagtctctccgtctttttaaactcttttttaagtttcccctcccctttcatattttttttcgccatttcttttagcattggactttggggtcgaaagcgtttctttttatttgcttcttttaagccgagcacagtttaggtttcgtgctgtcttaagagaactatccagcagcttcttgctcatccttattgggagaactgcaccgttactttaaaaacacacatacacaaaaaccttaagggagaaagcaggtaattgctgccatggaaacacaactgtctaatgggccaacttgcaataacacagccaatggtccaaccaccataaacaacaactgttcgtcaccagttgactctgggaacacagaagacagcaagaccaacttaatagtcaactaccttcctcagaacatgacacaggaggaactaaagagtctctttgggagcattggtgaaatagagtcctgtaagcttgtaagagacaaaataacagggcagagcttgggatatggctttgtgaactacattgaccccaaggatgcagagaaagctatcaacaccctgaatggattgagacttcaaaccaaaacaataaaagtttcctatgctcgcccaagttcagcttctatcagagatgcaaatttatatgtcagcggacttccaaaaacaatgacccagaaggagttggaacagcttttttcacaatatggacgcattattacttctcgtattcttgtcgaccaggtcactggcatatcaaggggtgtagggtttattcgatttgacaagcgaattgaggcagaagaagctatcaaaggcctaaatggccagaaacctcccggtgccacggagccaatcactgtaaagtttgctaataacccaagccaaaaaaccaatcaggccatcctttcccagctgtaccagtctccaaacagaaggtatccaggaccgctagctcagcaggcacagcgttttaggttggacaatctgctcaatatggcttatggagtaaagaggttttctccaatgaccattgacggaatgaccagtttggctggaattaatatccctgggcaccctggaacagggtggtgtatatttgtgtacaacctggctcctgacgcagatgagagtatcctgtggcaaatgtttgggccttttggagctgtcaccaatgtgaaggtcatccgtgactttaacaccaataaatgcaaaggttttggatttgtgactatgacaaactatgatgaggctgccatggcgatagctagcctcaatggataccgtctgggagacagagtactgcaggtctcctttaagacaaacaaaacgcacaaagcctaatgagctcttgtcctcagtccatttatatatgaaaactatacaacaaaggcaagttaagagaaactttatacattagtaaatgtctttgtaagtcagtgttgagatggggataaaatgactacttagcatcctaagaaatatgtgagattttttattgctagtatttgaattaaaacttcttaaatatcttttatgtttgaatatggacaagaggtacagggtttttacctgtcacattgcattctattgccttctttgaagaaggtggaccttttaaagtgtttcagctaagggaagacatttcttttctttttacataactgccttgaacctgtgagtaaatattgaggctttgtgttgtaattcttcagttggttgtgtcttttttttcccccctttttttcctttttctgattagctttgtgtttggtttacatttaaagcattgctgttatgtctgtttaagaaaagtattttgaagtttacatttttatttatgaagtttaaaacagtatttattttgtaattatgatttgggttggggaagggggggctacattataaacgcttattgtaagaatactggagaacttttcgtaaagcagtaccttgccaaagagataagagcctctttgatgtgggtttaaaaaaagcatctatttttataaaaaagaaaatttggagaaactttttactggtcctggaacaaatattttgacttgaatactttgagaaatctcttcatatgacacctagtgagcttttaaaatttaccaggaaatttgcagcggttggaaaatttagaaagatttatggtgtagaaaatacttttgagatctttgtatgaaaggagtagaatcaatggggggaaacactgctggtttcatttttgtaatcaccagtggagcgtctgatcatcctggttattatgtgataggtggctcacattgatttgtgattttgaaacaaataaaaaaaatttacaaaagaatatataagagcaggcaagaaatttaaattaccgagagatgggggaaaaaatctgttcttcctaaagaaatcccttcagatagagctcatggtgtttagtgatgtacttgcagtattgtttgaagaattgttttgtcttaaggaaaaaagacgttgcacatgatttgtactgcagcaaatcagcaaaagtgatctgagttggatatatttgaaggtattttgaaagttacgttcaaggctaacacctgagctttgtgtaatgtaaataagaccttgtgtttatgaacctttcagctaatttaattttttttcccttacatgccaagtgatgttcaggttttgaatgtttttgtatcagttttttcctttgtaaatggcattaacattgttacttgaggtcttgcttaatcacttttgttgtcctgaggacttgaatttacagtgcatcagatttgttgcaaattttgtctgtagatagtctagcttcagctgtttatggtgatgctacattttcgtttataaatatgtttgtggtataaaaaaatgagtataaccataggttttgaacaaatttccttacatttttcatacaaaaatcataaatatctgtatgctattgaaatttaactttgtatgatgcttaaaaaccactatttggggaaataataaaataagtctttaccatgtatgaaagaaattttaaaaaatacaaaatattttctgattagcatctagcttataataaattttcaaaaaagctgaaggcaaaaatgccttcatcaggatgcactgagaactatatagttacgtcctgctttttgtataaactgagatgctcacatgcttccccttagaacaggcaatgtgctatgcataacatagttgtacattatctttgcggttgctttgagttttattttttattatttaaaattgtagttataaaatttttcagtatagtacagtacatatactgtgaggcgcgtgctaaagtgaataagcgagttttcatgctgacccactcaatgctattcagaaatcaattggcttagcactttctcatatccttaggtgcatttagattgccagagttaaccttctgcgtttaaaaaaagaaaaacactaaaaaataaaatacatgtatatacttaaaaaaaaataataaggtttccctcaagggaaaacagcagctacatgcttctttcctatactactgtagcaaaccaaggcattgatgagagggcatgcaaattgtgcttcactttacagtgttttatcagagcacttaataaaatgtaaggctggtatttatttgaagttgtacagtatgacttaattcacatctgttggaatagaaaatatattctgttgagtatttaagaggctgtacatgttttcttttgtgtttggattctttgtactttttcatgttcagtacatcaataaacaaagttgaagggaaaaaaaaa

SEQ ID NO: 5 - DNA, 321 bp, Artificial Sequence
  SCN5A splice variant Exon 28B
ggagccctcctagtgagtatgaagtgatatctcactgaggttttggtttgcaaaagcaaatgactgatgactaacgatgcaggacatctttccatgtgcatgttggtcatttatatatcttccttggagaaatctctattcagatccttagctcatttttaattgggttatttctcttcttcttgttgagttgtaagagttctttacatattctggatcacagtctcttatcagatatatgatttaaaaatattttctcctagtctgtgagttttttcatttcctagtggtgtccattaaagcacaaaagttttacatgtt

SEQ ID NO: 6 - DNA, 164 bp, Artificial Sequence
  SCN5A splice variant Exon 28C
gaactgcacaatgaccagcaggaggggagaagagagtaggaaaaaggagggaaggacagacatcaagtgccagatgttgtctgaactaatcgagcacttctcaccaaacttcatgtataaataaaatacatatttttaaaacaaaccaataaatggcttacatg

SEQ ID NO: 7 - DNA, 114 bp, Artificial Sequence
  SCN5A splice variant Exon 28D
ggcactgtgctctcggacatcatccagaagtacttcttctccccgacgctcttccgagtcatccgcctggcccgaataggccgcatcctcagactgatccgaggggccaagggg

SEQ ID NO: 8 - protein, 473 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 9 - DNA, 6194 bp, Homo sapiens
aagggggcaaagcctcggtcttcatagaaaaggagaggaggcaaacgcagcccaaactggggggtttctcttcaaagccagctggtctggctttattctgcaggaatttttttacctgtcagggtttggacaacaaagccctcagcaggtgctgacgggtacaacttcctggagaagcagaaaggcactggtgccaaagaagagttgcaaactgtgaagtaacttctatgaagagatgaagtaaagaacggaaggcaaatgattgtggcagtaaagaagtgtatgtgcaggaacgaatgcaggaatttgggaactgagctgtgcaagtgctgaagaaggagatttgtttggaggaaacaggaaagagaaagaaaaggaaggaaaaaatacataatttcagggacgagagagagaagaaaaacggggactatggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggaggcattgaacaagaaagaaaacaaaggctgtgaaagccccgatcccgactcctcttatgcactcaccccacgcactgaagaaaaatacaaaaaaattaatgaagaatttgataatatgatcaagagtcataaaattcctgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatgatcagattattacttactagtttttttttttttcttgcagtgtgtgtgtgtgctataccttaatggggaaggggggtcgatatgcattatatgtgccgtgtgtggaaaaaaaaaaagtcaggtactctgttttgtaaaagtacttttaaattgcctcagtgatacagtataaagataaacagaaatgctgagataagcttagcacttgagttgtacaacagaacacttgtacaaaatagattttaaggctaacttcttttcactgttgtgctcctttgcaaaatgtatgttacaatagatagtgtcatgttgcaggttcaacgttatttacatgtaaatagacaaaaggaaacatttgccaaaagcggcagatctttactgaaagagagagcagctgttatgcaacatatagaaaaatgtatagatgcttggacagacccggtaatgggtggccattggtaaatgttaggaacacaccaggtcacctgacatcccaagaatgctcacaaacctgcaggcatatcattggcgtatggcactcattaaaaaggatcagagaccattaaaagaggaccatacctattaaaaaaaaatgtggagttggagggctaacatatttaattaaataaataaataaatctgggtctgcatctcttattaaataaaaatataaaaatatgtacattacattttgcttattttcatataaaaggtaagacagagtttgcaaagcatttgtggctttttgtagtttacttaagccaaaatgtgtttttttccccttgatagcttcgctaatattttaaacagtcctgtaaaaaaccaaaaaggactttttgtatagaaagcactaccctaagccatgaagaactccatgctttgctaaccaagataactgttttctctttgtagaagttttgtttttgaaatgtgtatttctaattatataaaatattaagaatcttttaaaaaaatctgtgaaattaacatgcttgtgtatagctttctaatatatataatattatggtaatagcagaagttttgttatcttaatagcgggaggggggtatatttgtgcagttgcacatttgagtaactattttctttctgttttcttttactctgcttacattttataagtttaaggtcagctgtcaaaaggataacctgtggggttagaacatatcacattgcaacaccctaaattgtttttaatacattagcaatctattgggtcaactgacatccattgtatatactagtttctttcatgctatttttattttgttttttgcatttttatcaaatgcagggcccctttctgatctcaccatttcaccatgcatcttggaattcagtaagtgcatatcctaacttgcccatattctaaatcatctggttggttttcagcctagaatttgatacgctttttagaaatatgcccagaatagaaaagctatgttggggcacatgtcctgcaaatatggccctagaaacaagtgatatggaatttacttggtgaataagttataaattcccacagaagaaaaatgtgaaagactgggtgctagacaagaaggaagcaggtaaagggatagttgctttgtcatccgtttttaattattttaactgacccttgacaatcttgtcagcaatataggactgttgaacaatcccggtgtgtcaggacccccaaatgtcacttctgcataaagcatgtatgtcatctattttttcttcaataaagagatttaatagccatttcaagaaatcccataaagaacctctctatgtccctttttttaatttaaaaaaaatgactcttgtctaatattcgtctataagggattaattttcagaccctttaataagtgagtgccataagaaagtcaatatatattgtttaaaagatatttcagtctaggaaagattttccttctcttggaatgtgaagatctgtcgattcatctccaatcatatgcattgacatacacagcaaagaagatataggcagtaatatcaacactgctatatcatgtgtaggacatttcttatccattttttctcttttacttgcatagttgctatgtgtttctcattgtaaaaggctgccgctgggtggcagaagccaagagaccttattaactaggctatatttttcttaacttgatctgaaatccacaattagaccacaatgcacctttggttgtatccataaaggatgctagcctgccttgtactaatgttttatatattaaaaaaaaaaaatctatcaaccatttcatatatatcccactactcaaggtatccatggaacatgaaagaataacatttatgcagaggaaaaacaaaaacatccctgaaaatatacacactcatacacacacacgcacaggggaataaaataagaaaatcattttcctcaccatagacttgatcccatccttacaacccatccttctaacttgatgtgtataaaatatgcaaacatttcacaaatgttctttgtcatttcaaaatactttagtatatcaatatcagtagataccagtgggtgggaaagggtcattacatgaaaatatgaagaaatagccatattagttttttaacctgcaatttgcctcagcaacaaagaaaaagtgaatttttaatgctgaagataaagtaagctaaagtaccagcagaagccttggctatttatagcagttctgacaatagttttataagaacatgaagagaacagaatcacttgaaaatggatgccagtcatctcttgttcccactactgaattcttataaagtggtggcaagatagggaagggataatctgagaatttttaaaagatgatttaatgagaagaagcacaattttgattttgatgagtcactttctgtaaacaatcttggtctatctttacccttataccttatctgtaatttaccatttattgtatttgcaaagctagtatggtttttaatcacagtaaatcctttgtattccagactttagggcagagccctgagggagtattattttacataacccgtcctagagtaacattttaggcaacattcttcattgcaagtaaaagatccataagtggcattttacacggctgcgagtattgttatatctaatcctattttaaaagatttttggtaatatgaagcttgaatactggtaacagtgatgcaatatacgcaagctgcacaacctgtatattgtatgcattgctgcgtggaggctgtttatttcaacctttttaaaaattgtgttttttagtaaaatggcttattttttcccaaaggtggaatttagcattttgtaatgatgaatataaaaatacctgtcatccccagatcatttaaaagttaactaaagtgagaatgaaaaaacaaaattccaagacactttttaaaagaatgtctgccctcacacacttttatggatttgtttttcttacatacccatcttttaacttagagatagcattttttgccctctttattttgttgtttgtttctccagagagtaaacgctttgtagttctttctttaaaaaacattttttttaaagaagaagaagccacttgaaccctcaataaaggctgttgcctaagcatggcatacttcatctgttctcatttgtgccatctgccgtgatgtcgtcacttttatggcgttaatttcctgccactacagatcttttgaagattgctggaatactggtgtctgttagaatgcttcagactacagatgtaattaaaggcttttcttaatatgttttaaccaaagatgtggagcaatccaagccacatatcttctacatcaaatttttccattttggttattttcataatctggtattgcattttgccttccctgttcatacctcaaattgattcatacctcagtttaattcagagaggtcagttaagtgacggattctgttgtggtttgaatgcagtaccagtgttctcttcgagcaaagtagacctgggtcactgtaggcataggacttggattgcttcagatggtttgctgtatcatttttcttctttttcttttcctggggacttgtttccattaaatgagagtaattaaaatcgcttgtaaatgagggcatacaagcatttgcaacaaatattcaaatagaggctcacagcggcataagctggactttgtcgccactagatgacaagatgttataactaagttaaaccacatctgtgtatctcaagggacttaattcagctgtctgtagtgaataaaagtgggaaattttcaaaagtttctcctgctggaaataaggtataatttgtattttgcagacaattcagtaaagttactggctttcttagtgaaaaaaaaaaa

SEQ ID NO: 10 - protein, 268 aa, Homo sapiens
MAQPLCPPLSESWMLSAAWGPTRRPPPSDKDCGRSLVSSPDSWGSTPADSPVASPARPGTLRDPRAPSVGRRGARSSRLGSGQRQSASEREKLRMRTLARALHELRRFLPPSVAPAGQSLTKIETLRLAIRYIGHLSAVLGLSEESLQRRCRQRGDAGSPRGCPLCPDDCPAQMQTRTQAEGQGQGRGLGLVSAVRAGASWGSPPACPGARAAPEPRDPPALFAEAACPEGQAMEPSPPSPLLPGDVLALLETWMPLSPLEWLPEEPK

SEQ ID NO: 11 - DNA, 1184 bp, Homo sapiens
tggaaggggccacttcacacctcgggctcggcataaagcggccgccggccgccggcccccagacgcgccgccgctgccatggcccagcccctgtgcccgccgctctccgagtcctggatgctctctgcggcctggggcccaactcggcggccgccgccctccgacaaggactgcggccgctccctcgtctcgtccccagactcatggggcagcaccccagccgacagccccgtggcgagccccgcgcggccaggcaccctccgggacccccgcgccccctccgtaggtaggcgcggcgcgcgcagcagccgcctgggcagcgggcagaggcagagcgccagtgagcgggagaaactgcgcatgcgcacgctggcccgcgccctgcacgagctgcgccgctttctaccgccgtccgtggcgcccgcgggccagagcctgaccaagatcgagacgctgcgcctggctatccgctatatcggccacctgtcggccgtgctaggcctcagcgaggagagtctccagcgccggtgccggcagcgcggtgacgcggggtcccctcggggctgcccgctgtgccccgacgactgccccgcgcagatgcagacacggacgcaggctgaggggcaggggcaggggcgcgggctgggcctggtatccgccgtccgcgccggggcgtcctggggatccccgcctgcctgccccggagcccgagctgcacccgagccgcgcgacccgcctgcgctgttcgccgaggcggcgtgccctgaagggcaggcgatggagccaagcccaccgtccccgctccttccgggcgacgtgctggctctgttggagacctggatgcccctctcgcctctggagtggctgcctgaggagcccaagtgacaagggacaactgacgccgtctctgtgagcaccgaggctttttggcctcagcaccttcgaagtggttccttggcagactgcctttcctggaagagggcacgggcgatcccgacgggggcattcctgcgggtgagagccgtccccaccgcggcggcccttctcagcccctccctccatggagggacccatagggctagacactttgaggcaagcaggaggctctgcctaatgtgaatttatttatttgtgaataaactgtactggtgtcagttggcaaaaaaaaaaaaaaaaaaaaaaaa

SEQ ID NO: 12 - protein, 2016 aa, Homo sapiens [sequence omitted]

SEQ ID NO: 13 - DNA, 8504 bp, Homo sapiens
agacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatga